(19) United States
(12) Patent Application Publication
(10) Pub. No.: US 2008/0312322 A1
(43) Pub. Date: Dec. 18, 2008

(54) Organic Compounds

(76) Inventors: Urs Baettig, West Sussex (GB); Brian Cox, West Sussex (GB); Katharine Louise Turner, West Sussex (GB); Simon James Watson, West Sussex (GB); Diana Janus, West Sussex (GB); Catherine Leblanc, West Sussex (GB); David Andrew Sandham, West Sussex (GB)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/915,091

(22) PCT Filed: May 22, 2006
(86) PCT No.: PCT/EP2006/004844

§ 371 Date: Jul. 8, 2008

(30) Foreign Application Priority Data
May 24, 2005 (GB) 0510585.3

(51) Int. Cl. A61K 31/216; C07C 59/40; A61K 31/19; A61P 37/00; A61P 29/00; C07C 69/76

(52) U.S. Cl. 514/533; 562/468; 560/57; 514/568

(57) ABSTRACT

A compound of formula (I); in free or pharmaceutically acceptable salt form, where R¹, R², R³, R⁴, R⁵, m, n, w, X, Y and Q have the meanings as indicated in the specification, are useful for treating conditions mediated by the CRTh₂ receptor, especially inflammatory or obstructive airways diseases.

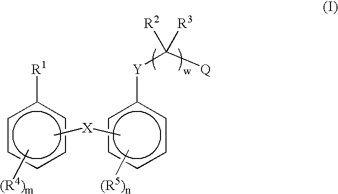

The present invention relates to organic compounds, their preparation and their use as pharmaceuticals.

In a first aspect, the present invention provides compounds of formula (I)

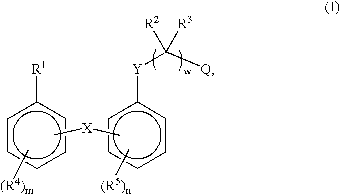

in free or pharmaceutically acceptable salt form,
wherein
Q is selected from —C(O)OR⁶, and —C(O)NR⁷R⁸;
R¹ is selected from OH, R^{1a}S—, R^{1a}O— and R^{1a}NR⁹—, wherein R^{1a} is

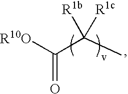

wherein R^{1b} and R^{1c} are, independently, H, C₁-C₈-alkyl, or together with the carbon atom to which they are attached form a divalent C₃-C₈-cycloaliphatic group;
R² and R³ are, independently, H, C₁-C₈-alkyl, or together with the carbon atom to which they are attached form a divalent C₃-C₈-cycloaliphatic group;
R⁴ and R⁵ are, independently, halogen, C₁-C₈-alkyl, C₁-C₈-haloalkyl, a C₃-C₁₅-carbocyclic group, nitro, cyano, C₁-C₈-alkylsulfinyl, C₁-C₈-alkylsulfonyl, C₁-C₈-haloalkylsulfonyl, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, C₁-C₈-alkylamino, di(C₁-C₈-alkyl)amino, SO₂NH₂, (C₁-C₈-alkylamino)sulfonyl, di(C₁-C₈-alkyl)aminosulfonyl, aminocarbonyl, C₁-C₈-alkylaminocarbonyl, di(C₁-C₈-alkyl)aminocarbonyl or a 4- to 10-membered heterocyclic group having one or more heteroatoms selected from the group consisting of oxygen, nitrogen and sulphur;
R⁶ is selected from H, C₁-C₈-alkyl, C₃-C₅-cycloalkyl, C₁-C₈-alkyl(C₆-C₁₅-aromatic carbocyclic group) and C₆-C₁₅-aromatic carbocyclic group;
R⁷ is H or C₁-C₈-alkyl;
R⁸ is C₃-C₁₅-cycloalkyl;
R⁹ and R¹⁰ are, independently selected from H, C₁-C₈-alkyl, C₃-C₁₅-cycloalkyl, C₁-C₈-alkyl(C₆-C₁₅-aromatic carbocyclic group), and C₆-C₁₅-aromatic carbocyclic group;
X is —CH₂—, —CH(C₁-C₈-alkyl)-, —CO—, —CH(OH)—, —CH(OC₁-C₈-alkyl)-, —C(halogen)₂-, —O—, —S—, —SO— or —SO₂—;
Y is —O—, —S—, —CH₂— or —NR¹¹(C₁-C₈-alkyl)-;
R¹¹ is selected from H, C₁-C₈-alkyl, C₃-C₁₅-cycloalkyl, C₁-C₈-alkyl(C₆-C₁₅-aromatic carbocyclic group), and C₆-C₁₅-aromatic carbocyclic group;
m and n are each, independently, an integer selected from 0-3;
v is an integer selected from 1-3; and
w is an integer selected from 0-3,
   with the proviso that said compound of formula (I) is not [2-(2-carboxymethoxy-5-chloro-benzyl)-4-chloro-phenoxy]-acetic acid, [2-(2-carboxymethoxy-5-methyl-benzyl)-4-methyl-phenoxy]-acetic acid, 2-{2-[2-(1-carboxy-1-methyl-ethoxy)-5-chloro-benzyl]-4-chloro-phenoxy}-2-methyl-propionic acid, 2-{2-[(1-carboxyethoxy)-5-chloro-3-methyl-benzyl]-4-chloro-6-methyl-phenoxy}propionic acid, 3′,3′-[methylenebis[(4-methyl-2,1-phenylene)bis-propanoic acid, 2,2′-[methylenebis[[4-(1,1-dimethylethyl)-2,1-phenylene]oxy]]bis-acetic acid, diethyl ester, 2,2′-[methylenebis[(3,4,6-trichloro-2,1-phenylene)oxy]]bis-acetic acid, 4-[4-chloro-2-[(5-chloro-2-hydroxyphenyl)methyl]phenoxy]-butanoic acid, monosodium salt, 4-[4-chloro-2-[(4-chloro-2,1-phenylene)oxy]]-butanoic acid, disodium salt, [4-chloro-2-[(4-chloro-2-hydroxyphenyl)methyl]phenoxy]-acetic acid, 2,2′-[methylenebis[(4-chloro-2,1-phenylene)oxy]]bis-acetic acid, [thiobis[(4,6-dichloro-o-phenylene)oxy]di-acetic acid, 3,3′-[methylenebis[(3,4,6-trichloro-o-phenylene)oxy]]di-propionic acid, 2,2′-[methylenebis[(4-methyl-2,1-phenylene)oxy]]bis-acetic acid, or 2,2′-[methylenebis[(4-methyl-2,1-phenylene)oxy]]bis-acetic acid, diethyl ester.

Where in formula (I) m or n is 2, the two moieties R⁴ or two moieties R⁵ may be the same or different. Where m or n is 3, two or all of the three moieties R⁴ or the three moieties R⁵ may be the same or all three may be different.

According to formula (I), Q is selected from —C(O)OR⁶, where R⁶ is suitably H or C₁-C₈-alkyl, such as methyl, ethyl, isopropyl, and isobutyl.

According to formula (I), Q is selected from —C(O)NR⁷R⁸, where R⁷ is suitably H and R⁸ is suitably C₃-C₁₅ cycloalkyl, such as a cyclopropyl ring.

According to formula (I), R¹ is selected from OH and

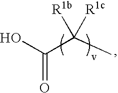

where R^{1b} and R^{1c} are independently selected from H and C₁-C₈-alkyl.

According to formula (I), R² and R³ are suitably H.

According to formula (I), R⁴ and R⁵ are selected from H, halogen, nitro, and C₁-C₈-alkyl.

According to formula (I), X is suitably —CH₂—, —CH(C₁-C₈-alkyl)-, —CO—, —CH(OH)—, —CH(OC₁-C₈-alkyl)-, —C(halogen)₂-, —O—, —S—, —SO— or —SO₂—.

According to formula (I), Y is —O—, —S—, —CH₂— or —NR¹¹(C₁-C₈-alkyl)-, where
R¹¹ is selected from H, C₁-C₈-alkyl, C₃-C₁₅-cycloalkyl, C₁-C₈-alkyl(C₆-C₁₅-aromatic carbocyclic group), and C₆-C₁₅-aromatic carbocyclic group.
m and n are each, suitably 1.

According to formula (I), v is an integer selected from 1-3. According to formula (I), w is an integer selected from 1.

Another embodiment of the present invention provides compounds in free or pharmaceutically acceptable salt form, wherein the compound is of formula (Ia)

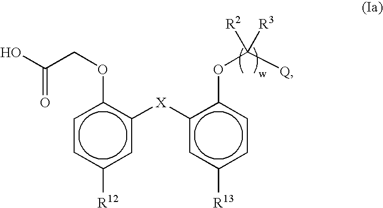

wherein
Q is selected from —C(O)OR⁶, and —C(O)NR⁷R⁸;
R² and R³ are H;
R⁶ is H or C₁-C₈-alkyl, such as methyl, ethyl, isopropyl, and isobutyl;
R⁷ is H;
R⁸ is C₃-C₁₅ cycloalkyl, such as a cyclopropyl ring;
R¹² and R¹³ are, independently, H, halogen, nitro, or C₁-C₈-alkylsulfonyl, such as —SO₂CH₃;
X is —CH₂—, S, —SO— or —SO₂—, preferably —CH₂—; and
w is 1.

In another embodiment, the present invention provides for the use of a compound of formula (I) in any of the aforementioned embodiments, in free or pharmaceutically acceptable salt form, for the manufacture of a medicament for the treatment of an inflammatory or allergic condition, particularly an inflammatory or obstructive airways disease.

It is understood that any and all embodiments of the present invention may be taken in conjunction with any other embodiment to describe additional embodiments of the present invention. Furthermore, any elements of an embodiment are meant to be combined with any and all other elements from any of the embodiments to describe additional embodiments.

### DEFINITIONS

Terms used in the specification have the following meanings:

“Optionally substituted”, as used herein, means the group referred to can be substituted at one or more positions by any one or any combination of the radicals listed thereafter.

“Halogen” or “halo” may be fluorine, chlorine, bromine or iodine; preferably it is bromine or chlorine or fluorine.

“C₁-C₈-Alkyl” denotes straight-chain or branched C₁-C₈-alkyl, which may be, e.g., methyl, ethyl, n-propyl, isopropyl, n-butyl, isobutyl, sec-butyl, tert-butyl, straight- or branched-pentyl, straight- or branched-hexyl, straight- or branched-heptyl or straight- or branched-octyl. Preferably, C₁-C₈-alkyl is C₁-C₄-alkyl.

“C₃-C₁₅-Carbocyclic group”, as used herein, denotes a carbocyclic group having 3- to 15-ring carbon atoms, e.g., a monocyclic group, either cycloaliphatic, such as a C₃-C₈-cycloalkyl, e.g., cyclopropyl, cyclobutyl, cyclopentyl, cyclohexyl, cycloheptyl or cyclooctyl; or aromatic, such as phenyl; or a bicyclic group, such as bicyclooctyl, bicyclononyl including indanyl and indenyl, and bicyclodecyl including naphthyl. Preferably the C₃-C₁₅-carbocyclic group is a C₃-C₁₀-carbocyclic group, e.g., phenyl or naphthyl. The C₃-C₁₅-carbocyclic group can be substituted with 1-3 substituents or unsubstituted. Preferred substituents include halo, cyano, amino, nitro, carboxy, C₁-C₈-alkyl, C₁-C₈-haloalkyl, C₁-C₈-alkoxy, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₀-carbocyclic group and a 5- to 12-membered heterocyclic group having at least one ring heteroatom selected from nitrogen, oxygen and sulphur.

“C₆-C₁₅-Aromatic carbocyclic group”, as used herein, denotes a divalent aromatic group having 6- to 15-ring carbon atoms, e.g., phenylene, naphthylene or anthrylene. The C₆-C₁₅-aromatic group can be substituted with 1-3 substituents or can be unsubstituted. Preferred substituents include halo, cyano, amino, nitro, carboxy, C₁-C₈-alkyl, halo-C₁-C₈-alkyl, C₁-C₈-alkoxy, C₁-C₈-alkylcarbonyl, C₁-C₈-alkoxycarbonyl, C₁-C₈-haloalkoxy, carboxy-C₁-C₈-alkyl, C₁-C₈-alkylamino, di(C₁-C₈-alkylamino), C₁-C₈-alkylsulfonyl, —SO₂NH₂, (C₁-C₈-alkylamino)sulfonyl, di(C₁-C₈-alkyl)aminosulfonyl, aminocarbonyl, C₁-C₈-alkylaminocarbonyl and di(C₁-C₈-alkyl)aminocarbonyl, a C₃-C₁₅-carbocyclic group and a 5- to 12-membered heterocyclic group having at least one ring heteroatom selected from nitrogen, oxygen and sulphur.

“Divalent C₃-C₈-cycloaliphatic” denotes cycloalkylene having 3- to 8-ring carbon atoms, e.g., a monocyclic group, such as a cyclopropylene, cyclobutylene, cyclopentylene, cyclohexylene, cycloheptylene or cyclooctylene, any of which can be substituted by one or more, usually one or two, C₁-C₄-alkyl groups; or a bicyclic group, such as bicycloheptylene or bicyclooctylene. Preferably “C₃-C₈-cycloalkylene” is C₃-C₅-cycloalkylene, e.g., cyclopropylene, cyclobutylene or cyclopentylene.

“C₁-C₈-Alkoxy” denotes straight-chain or branched C₁-C₈-alkoxy which may be, e.g., methoxy, ethoxy, n-propoxy, isopropoxy, n-butoxy, isobutoxy, sec-butoxy, tert-butoxy, straight- or branched-pentoxy, straight- or branched-hexyloxy, straight- or branched-heptyloxy or straight- or branched-octyloxy. Preferably, C₁-C₈-alkoxy is C₁-C₄-alkoxy.

“C₁-C₈-Haloalkyl” and “C₁-C₈-haloalkoxy” denote C₁-C₈-alkyl and C₁-C₈-alkoxy, as hereinbefore defined, substituted by one or more halogen atoms, preferably one, two or three halogen atoms, preferably fluorine, bromine or chlorine atoms. Preferably, C₁-C₈-haloalkyl is C₁-C₄-alkyl substituted by one, two or three fluorine, bromine or chlorine atoms. Preferably, C₁-C₈-haloalkoxy is C₁-C₄-alkoxy substituted by one, two or three fluorine, bromine or chlorine atoms.

“C₁-C₈-Alkylsulfonyl”, as used herein, denotes C₁-C₈-alkyl, as hereinbefore defined, linked to —SO₂—. Preferably, C₁-C₈-alkylsulfonyl is C₁-C₄-alkylsulfonyl, especially methylsulfonyl.

“Amino-C₁-C₈-alkyl” and “amino-C₁-C₈-alkoxy” denote amino attached by a nitrogen atom to C₁-C₈-alkyl, e.g., NH₂—(C₁-C₈)—, or to C₁-C₈-alkoxy, e.g., NH₂—(C₁-C₈)—O—, respectively, as hereinbefore defined. Preferably, amino-C₁-C₈-alkyl and amino-C₁-C₈-alkoxy are, respectively, amino-C₁-C₄-alkyl and amino-C₁-C₄-alkoxy.

“Amino-(hydroxy)-C₁-C₈-alkyl” denotes amino attached by a nitrogen atom to C₁-C₈-alkyl and hydroxy attached by an oxygen atom to the same C₁-C₈-alkyl. Preferably, amino-(hydroxy)-C₁-C₈-alkyl is amino-(hydroxy)-C₂-C₄-alkyl.

“Carboxy-C₁-C₈-alkyl” and “carboxy-C₁-C₈-alkoxy” denote carboxy attached by a carbon atom to C₁-C₈-alkyl or C₁-C₈-alkoxy, respectively, as hereinbefore defined. Preferably, carboxy-C₁-C₈-alkyl and carboxy-C₁-C₈-alkoxy are, respectively, carboxy-C₁-C₄-alkyl and carboxy-C₁-C₄-alkoxy.

“C₁-C₈-Alkylcarbonyl”, “C₁-C₈-alkoxycarbonyl” and “C₁-C₈-haloalkylcarbonyl” denote C₁-C₈-alkyl, C₁-C₈-alkoxy or C₁-C₈-haloalkyl, respectively, as hereinbefore defined, attached by a carbon atom to a carbonyl group. “C₁-C₈-Alkoxycarbonyl” denotes C₁-C₈-alkoxy, as hereinbefore defined, wherein the oxygen of the alkoxy group is attached to the carbonyl carbon. Preferably, C₁-C₈-alkylcarbonyl, C₁-C₈-alkoxycarbonyl and C₁-C₈-haloalkylcarbonyl are, respectively, C₁-C₄-alkylcarbonyl, C₁-C₄-alkoxycarbonyl and C₁-C₄-haloalkylcarbonyl.

“C₁-C₈-Alkylamino” and “di(C₁-C₈-alkyl)amino” denote C₁-C₈-alkyl, as hereinbefore defined, attached by a carbon atom to an amino group. The C₁-C₈-alkyl groups in di(C₁-C₈-alkyl)amino may be the same or different. Preferably, C₁-C₈-alkylamino and di(C₁-C₈-alkyl)amino are, respectively, C₁-C₄-alkylamino and di(C₁-C₄-alkyl)amino.

“C₁-C₈-Alkylaminocarbonyl” and “di(C₁-C₈-alkyl)aminocarbonyl” denote C₁-C₈-alkylamino and di(C₁-C₈-alkyl)amino, respectively, as hereinbefore defined, attached by a nitrogen atom to the carbon atom of a carbonyl group. Preferably, C₁-C₈-alkylaminocarbonyl and di(C₁-C₈-alkyl)-aminocarbonyl are, respectively, C₁-C₄-alkylaminocarbonyl and di(C₁-C₄-alkyl)-aminocarbonyl.

“4- to 10-membered heterocyclic group containing at least one ring heteroatom selected from the group consisting of nitrogen, oxygen and sulphur”, as used herein, may be moncyclic or bicyclic, e.g., furan, tetrahydrofuran, pyrrole, pyrrolidine, pyrazole, imidazole, triazole, isotriazole, tetrazole, thiadiazole, isothiazole, oxadiazole, pyridine, oxazole, isoxazole, pyrazine, pyridazine, pyrimidine, piperidine, piperazine, morpholine, triazine, oxazine, thiazole, quinoline, isoquinoline, benzothiophene, benzoxazole, benzisoxazole, benzothiazole, benzisothiazole, benzofuran, indole, indazole or benzimidazole. Preferred heterocyclic groups include piperazine, morpholine, imidazole, isotriazole, pyrazole, pyridine, furan, oxazole, oxadiazole, isoxazole, thiazole, tetrazole benzothiophene, benzoxazole, benzothiazole and benzofuran. The 4- to 10-membered heterocyclic group can be unsubstituted or substituted. Preferred substituents include halo, cyano, oxo, hydroxy, carboxy, nitro, C₁-C₈-alkyl, C₁-C₈-alkylcarbonyl, hydroxy-C₁-C₈-alkyl, C₁-C₈-haloalkyl, amino-C₁-C₈-alkyl, amino(hydroxy)C₁-C₈-alkyl and C₁-C₈-alkoxy optionally substituted by aminocarbonyl. Especially preferred substituents include halo, oxo, C₁-C₄-alkyl, C₁-C₄-alkylcarbonyl, hydroxy-C₁-C₄-alkyl, C₁-C₄-haloalkyl, amino-C₁-C₄-alkyl and amino(hydroxy)C₁-C₄-alkyl.

Throughout this specification and in the claims that follow, unless the context requires otherwise, the word “comprise”, or variations, such as “comprises” or “comprising”, will be understood to imply the inclusion of a stated integer or step or group of integers or steps but not the exclusion of any other integer or step or group of integers or steps.

Many of the compounds represented by formula (I) are capable of forming acid addition salts, particularly pharmaceutically acceptable acid addition salts. Pharmaceutically acceptable acid addition salts of the compound of formula (I) include those of inorganic acids, e.g., hydrohalic acids, such as hydrochloric acid or hydrobromic acid; nitric acid; sulphuric acid; phosphoric acid; and organic acids, e.g., aliphatic monocarboxylic acids, such as formic acid, acetic acid, diphenylacetic acid, triphenylacetic acid, caprylic acid, dichloroacetic acid, trifluoroacetic acid, hippuric acid, propionic acid and butyric acid; aliphatic hydroxy acids, such as lactic acid, citric acid, gluconic acid, mandelic acid, tartaric acid or malic acid; dicarboxylic acids, such as adipic acid, aspartic acid, fumaric acid, glutamic acid, maleic acid, malonic acid, sebacic acid or succinic acid; aromatic carboxylic acids, such as benzoic acid, p-chlorobenzoic acid, or nicotinic acid; aromatic hydroxy acids, such as o-hydroxybenzoic acid, p-hydroxybenzoic acid, 1-hydroxynaphthalene-2-carboxylic acid or 3-hydroxynaphthalene-2-carboxylic acid; and sulfonic acids, such as ethanesulfonic acid, ethane-1,2-disulfonic acid, 2-hydroxyethanesulfonic acid, methanesulfonic acid, (+)-camphor-10-sulfonic acid, benzenesulfonic acid, naphthalene-2-sulfonic acid, naphthalene-1,5-disulfonic acid or p-toluenesulfonic acid. These salts may be prepared from compounds of formula (I) by known salt-forming procedures.

Compounds of formula (I) which contain acidic, e.g., carboxyl, groups, are also capable of forming salts with bases, in particular, pharmaceutically acceptable bases, such as those well-known in the art; suitable such salts include metal salts, particularly, alkali metal or alkaline earth metal salts, such as sodium, potassium, magnesium, calcium or zinc salts; or salts with ammonia or pharmaceutically acceptable organic amines or heterocyclic bases, such as benethamine, benzathine, diethanolamine, ethanolamine, 4-(2-hydroxyethyl)morpholine, 1-(2-hydroxyethyl)pyrrolidine, N-methyl glucamine, piperazine, triethanolamine or tromethamine. These salts may be prepared from compounds of formula (I) by known salt-forming procedures.

In those compounds where there is an asymmetric carbon atom or an axis of chirality the compounds exist in individual optically active isomeric forms or as mixtures thereof, e.g., as racemic or diastereomeric mixtures. The present invention embraces both individual optically active R and S isomers, as well as mixtures, e.g., racemic or diastereomeric mixtures thereof.

Specific especially preferred compounds of formula (I) include those hereinafter described in the Examples.

Since prodrugs are known to enhance numerous desirable qualities of pharmaceuticals, e.g., solubility, bioavailability, manufacturing, etc., the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers which release an active parent drug of the present invention in vivo when such prodrug is administered to a mammalian subject. Prodrugs of the present invention are prepared by modifying functional groups present in the compound in such a way that the modifications are cleaved, either in routine manipulation or in vivo, to the parent compound. Prodrugs include compounds of the present invention wherein a hydroxy, amino or sulfhydryl group is bonded to any group that, when the prodrug of the present invention is administered to a mammalian subject, it cleaves to form a free hydroxyl, free amino or free sulfhydryl group, respectively. Examples of prodrugs include, but are not limited to, acetate, formate and benzoate derivatives of alcohol and amine functional groups in the compounds of the present invention.

“Therapeutically effective amount” is intended to include an amount of a compound of the present invention alone or an amount of the combination of compounds claimed or an amount of a compound of the present invention in combination with other active ingredients effective to treat the inflammatory diseases described herein.

As used herein, “treating” or “treatment” cover the treatment of a disease-state in a mammal, particularly in a human, and include:
(a) preventing the disease-state from occurring in a mammal, in particular, when such mammal is predisposed to the disease-state but has not yet been diagnosed as having it;
(b) inhibiting the disease-state, i.e., arresting it development; and/or
(c) relieving the disease-state, i.e., causing regression of the disease state.

### Synthesis

Another embodiment of the present invention provides a process for the preparation of compounds of formula (I), in free or pharmaceutically acceptable salt form, which comprises the steps of:
(i) (A) for the preparation of compounds of formula (I),
   wherein
      R¹ is R^{1a}S—, R^{1a}O— or R^{1a}NR⁹, where R^{1a} is

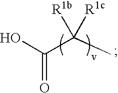

and
all other symbols are as hereinbefore defined,
cleaving an ester group —COOR¹⁰ in a compound of formula (I),
wherein
   R¹ is R^{1a}S—, R^{1a}O— or R^{1a}NR⁹,
   where
      R^{1a} is

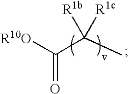

and
R¹⁰ is selected from C₁-C₈ alkyl, C₃-C₁₅-cycloalkyl, C₁-C₈-alkyl(C₆-C₁₅-aromatic carbocyclic group) and C₆-C₁₅-aromatic carboxylic group; and
all other symbols are as hereinbefore defined;
(B) for the preparation of compounds of formula (I),
wherein
   R¹ is OH;
   Q is —COOH; and
   all other symbols are as hereinbefore defined,
appropriately cleaving an ester group in a compound of formula (I),
wherein
   Q is —COOR⁶;
   R¹ is OH;
   R⁶ is C₁-C₈-alkyl, C₃-C₅-cycloalkyl, C₁-C₈-alkyl(C₆-C₁₅-aromatic carbocyclic group), or C₆-C₁₅-aromatic carboxylic group; and
   all other symbols are as hereinbefore defined; or
(C) for the preparation of compounds of formula (I),
wherein
   Q is —COOR⁶ or —C(O)NR⁷R⁸;
   R⁶ is C₁-C₈-alkyl, C₃-C₅-cycloalkyl, C₁-C₈-alkyl(C₆-C₁₅-aromatic carbocyclic group), and C₆-C₁₅-aromatic carboxylic group; and
   R⁷ and R⁸ are, as hereinbefore defined, appropriately esterifying or amidifying a compound of formula (I), where Q is —COOH; and
(ii) recovering the resultant compound of formula (I), in free or pharmaceutically acceptable salt form.

Process variants (A), (B) and (C) may be carried out using known procedures for ester cleavage or analogously as hereinafter described in the Examples.

Process variant (C) may be carried out using known procedures for conversion of a carboxylic acid to an ester or amide of the acid or analogously, as hereinafter described in the Examples.

Starting materials for process variants (A)-(C), and compounds for the preparation of those starting materials, may be novel or known; they may be prepared in accordance with known procedures or analogously, as hereinafter described in the Examples.

Another embodiment of the present invention provides compounds of formula (III)

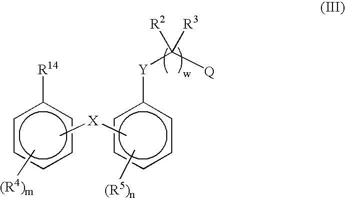

in free or pharmaceutically acceptable salt form, wherein
Q is —C(O)OR⁶,
R² and R³ are, independently, H, C₁-C₈-alkyl or together with the carbon atom to which they are attached form a divalent C₃-C₈-cycloaliphatic group;
R⁴ and R⁵ are, independently, halogen, C₁-C₈-alkyl, C₁-C₈-haloalkyl, a C₃-C₁₅-carbocyclic group, nitro, cyano, C₁-C₈-alkylsulfonyl, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, C₁-C₈-alkylamino, di(C₁-C₈-alkyl)amino, SO₂NH₂, (C₁-C₈-alkylamino)sulfonyl, di(C₁-C₈-alkyl)aminosulfonyl, aminocarbonyl, C₁-C₈-alkylaminocarbonyl, di(C₁-C₈-alkyl)aminocarbonyl or a 4- to 10-membered heterocyclic group having one or more heteroatoms selected from the group consisting of oxygen, nitrogen and sulphur;
R⁶ is selected from H, C₁-C₈-alkyl, C₃-C₅-cycloalkyl, C₁-C₈-alkyl(C₆-C₁₅-aromatic carbocyclic group) and C₆-C₁₅-aromatic carbocyclic group;
X is —CH₂—, —CO—, —CH(OH)—, —CH(OC₁-C₈-alkyl)-, —C(halogen)₂-, —O—, —S—, —SO— or —SO₂—;
Y is —O—, —S—, —CH₂— or —NR¹¹(C₁-C₈-alkyl);
R¹¹ is selected from H, C₁-C₈-alkyl, C₃-C₁₅-cycloalkyl, C₁-C₈-alkyl(C₆-C₁₅-aromatic carbocyclic group) and C₆-C₁₅-aromatic carbocyclic group;
R¹⁴ is —(CR^{1b}R^{1c})ₚCN, wherein R^{1b} and R^{1c} are, independently, H or C₁-C₈-alkyl;
m and n are each, independently, an integer selected from 0-3;
p is an integer selected from 0-2; and
w is an integer selected from 0-3.

Compounds of formula (III) may be used to prepare compounds of formula (I), wherein Q is a heterocycle, particularly tetrazole, in accordance with known procedures or analogously as hereinafter described in the Examples or Scheme 4.

Compounds of formula (III),

wherein
Q is —COOR⁶; and
R⁶ is C₁-C₈-alkyl, may be prepared by reacting a compound of formula (IV)

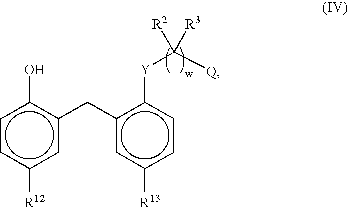

with a corresponding haloalkyl nitrile,
wherein
   R⁶ is C₁-C₈-alkyl; and
   R¹² and R¹³ are, independently, H, halogen, C₁-C₈-alkyl, C₁-C₈-haloalkyl, a C₃-C₁₅-carbocyclic group, nitro, cyano, C₁-C₈-alkylsulfinyl, C₁-C₈-alkylsulfonyl, C₁-C₈-haloalkylsulfonyl C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, C₁-C₈-alkylamino, di(C₁-C₈-alkyl)amino, SO₂NH₂, (C₁-C₈-alkylamino)sulfonyl, di(C₁-C₈-alkyl)aminosulfonyl, aminocarbonyl, C₁-C₈-alkylaminocarbonyl, di(C₁-C₈-alkyl)aminocarbonyl or a 4- to 10-membered heterocyclic group having one or more heteroatoms selected from the group consisting of oxygen, nitrogen and sulphur; and
all other symbols are as hereinbefore defined.

The compounds of formula (I) can be prepared, e.g., using the reactions and techniques described below. The reactions may be performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the invention.

The various substituents on the synthetic intermediates and final products shown in the following reaction schemes can be present in their fully elaborated forms, with suitable protecting groups where required as understood by one skilled in the art, or in precursor forms which can later be elaborated into their final forms by methods familiar to one skilled in the art. The substituents can also be added at various stages throughout the synthetic sequence or after completion of the synthetic sequence. In many cases, commonly used functional group manipulations can be used to transform one intermediate into another intermediate, or one compound of formula (I) into another compound of formula (I). Examples of such manipulations are conversion of an ester or a ketone to an alcohol; conversion of an ester to a ketone; interconversions of esters, acids and amides; alkylation, acylation and sulfonylation of alcohols and amines; and many others. Substituents can also be added using common reactions, such as alkylation, acylation, halogenation or oxidation. Such manipulations are well-known in the art, and many reference works summarize procedures and methods for such manipulations. Some reference works which gives examples and references to the primary literature of organic synthesis for many functional group manipulations, as well as other transformations commonly used in the art of organic synthesis are March's Organic Chemistry, 5^{th} Edition, Wiley and Chichester, Eds. (2001); Comprehensive Organic Transformations, Larock, Ed., VCH (1989); Comprehensive Organic Functional Group Transformations, Katritzky et al. (series editors), Pergamon (1995); and Comprehensive Organic Synthesis, Trost and Fleming (series editors), Pergamon (1991).

Generally, compounds described in the scope of this patent application can be synthesized by the routes described in Schemes 1-3.

In Scheme 1, condensation of a substituted phenol 1 with formaldehyde (preferably in the presence of a mineral acid, such as, but not limited to, H₂SO₄ to generate intermediate 2. Bis-alkylation of intermediate 2 with a haloacetate ester (preferably ethyl bromo acetate) in the presence of an inorganic base provides intermediate 3. Subsequent hydrolysis of intermediate 3 with an aqueous inorganic base provides the carboxylic acid derivative 4 which is selectively activated and then treated with an alcohol or amine to provide compound 5 in accordance with the procedure in J Org Chem, Vol. 58, p. 7948 (1993).

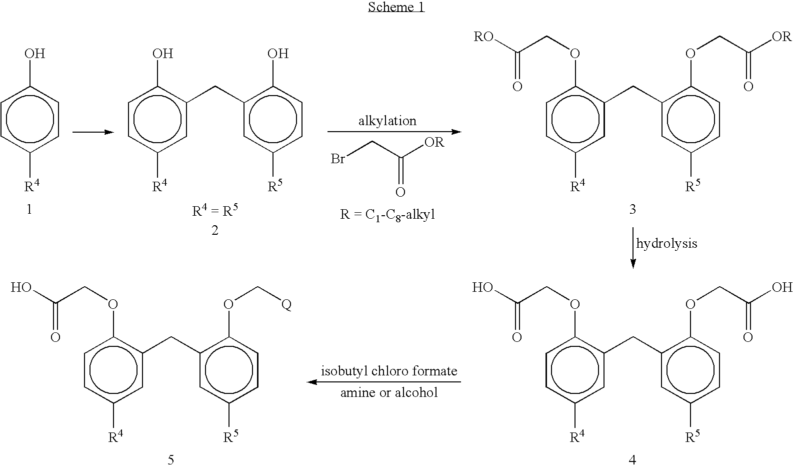

Scheme 2 also demonstrates another method for the preparation of compounds of formula (I). This method involves the cross-coupling of a benzyl halide, i.e., a benzyl bromide 6 with an aromatic boronic acid 7 and a palladium catalyst to generate intermediate 8. De-methylation of 8 and subsequent bis-alkylation with a haloacetate and hydrolysis yields compound 9.

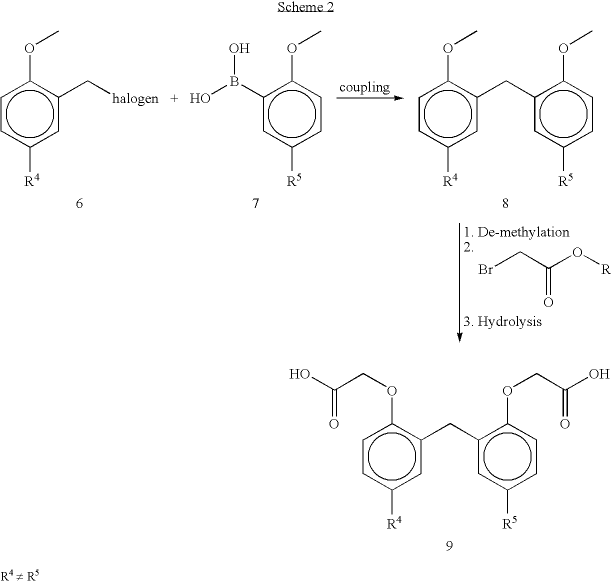

As outlined in the previous Schemes 1 and 2, compounds diaryl sulphide 10 and diaryl sulfone 12 can be transformed into respective compounds II and 13 or alternately into respective compounds 14 and 15, as shown in Scheme 3, below.

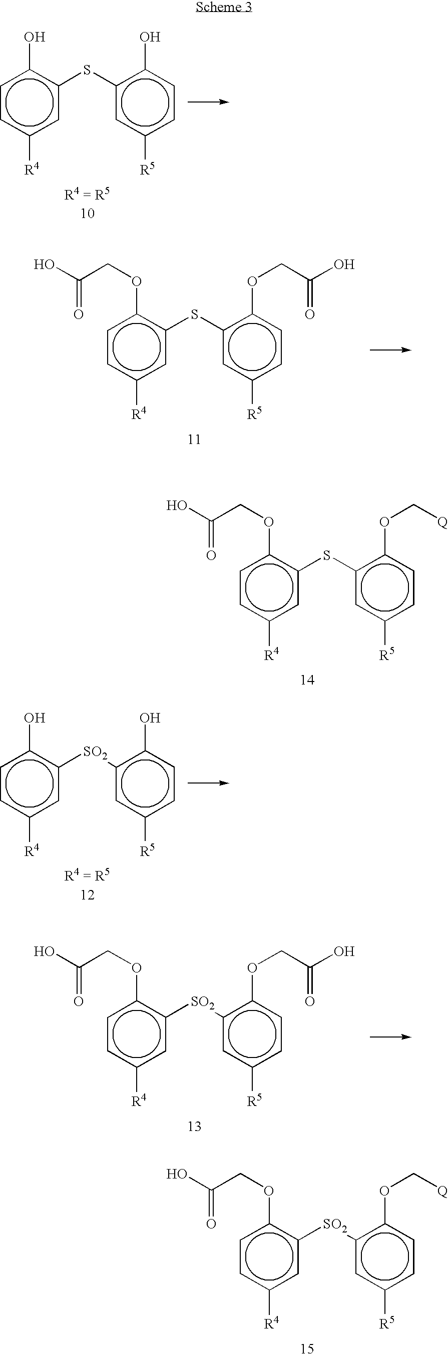

In an additional method to prepare compounds with R² and R³ both CH₃, phenols, such as 2 (Scheme 1) are treated with acetone and chloroform in the presence of an inorganic base, such as NaOH as in J Med Chem, Vol. 32, pp. 2460-2467 (1989).

In an additional method to prepare compounds with X═CO, diesters, such as 3 (Scheme 1) are treated with chromium trioxide in the presence of a carboxylic acid, such as acetic acid as in J Org Chem, Vol. 8, pp. 316-319 (1943), followed by ester hydrolysis.

### EXAMPLES

### General Conditions

LCMS are recorded on an Agilent 1100 LC system with a Waters Xterra MS C18 4.6×100 5 μM column, eluting with 5-95% 10 mM aqueous ammonium bicarbonate in acetonitrile over 2.5 minutes, with negative ion electrospray ionization or 5-95% water+0.1% TFA in acetonitrile with positive ion electrospray ionization. MH⁺ and [M−H]⁻ refer to monoisotopic molecular weights.

Melting points (m.p.) are uncorrected.

NMR are recorded at 400 MHz in CDCl₃, unless otherwise noted.

Abbreviations

| | |
|---|---|
| CHCl₃ | chloroform |
| DCM | dichloromethane |
| DIPEA | diisopropylethylamine |
| DMAP | 4-(dimethylamino)pyridine |
| DMF | dimethylformamide |
| EtOAc | ethyl acetate |
| HATU | O-(7-azabenzotriazol-1-yl)- |
| | N,N,N′,N′-tetramethyluronium |
| | hexafluorophophate |
| HCl | hydrochloric acid |
| HPLC | high performance liquid |
| | chromatography |
| MeOH | methanol |
| MgSO₄ | magnesium sulfate |
| NaHCO₃ | sodium bicarbonate |
| NaH | sodium hydride |
| NaOH | sodium hydroxide |
| Na₂SO₄ | sodium sulfate |
| Pd(PPh₃)₄ | tetrakis(triphenylphosphine) |
| | palladium(0) |
| TEA | triethylamine |
| TFA | trifluoroacetic acid |
| THF | tetrahydrofuran |

### Example 1

### a) Preparation of [4-chloro-2-(5-chloro-2-methoxycarbonylmethoxy-benzyl)-phenoxy]-acetic acid methyl ester

Cesium carbonate (60 g, 184 mmol) is added portionwise to a cooled (0° C.) solution of 2,2′-methylene-bis(4-chlorophenol) (24.48 g, 91 mmol) in DMF (120 mL), followed by methyl bromoacetate (17.3 mL, 184 mmol). The reaction is stirred at ambient temperature for 16 hours, then poured into 1 M aqueous HCl (1,000 mL) with ice bath cooling. The resultant solid collected by filtration, washed with water, re-crystallized from ethanol and dried in vacuo to afford [4-chloro-2-(5-chloro-2-methoxycarbonylmethoxy-benzyl)-phenoxy]-acetic acid methyl ester.

¹H NMR: δ 3.81 (6H, s), 4.05 (2H, s), 4.65 (4H, s), 6.78 (2H, d, J=8.7), 7.12 (2H, dd, J=2.2-8.7), 7.21 (2H, s).

### b) Preparation of 2-(2-carboxymethoxy-5-chloro-benzyl)-4-chloro-phenoxy]-acetic acid

2 M aqueous NaOH (100 mL) is added to a suspension of [4-chloro-2-(5-chloro-2-methoxycarbonylmethoxy-benzyl)-phenoxy]-acetic acid methyl ester (23.5 g, 56.9 mmol) in THF (100 mL) and the reaction stirred at ambient temperature overnight. The organic solvent is evaporated, the residue is diluted with water and acidified to pH 1 with concentrated HCl. The resultant solid is collected by filtration, washed with water and dried in vacuo to afford 2-(2-carboxymethoxy-5-chloro-benzyl)-4-chloro-phenoxy]-acetic acid; MH⁺=386.

### c) Preparation of [4-chloro-2-(5-chloro-2-methoxycarbonylmethoxy-benzyl)-phenoxy]-acetic acid

Isobutyl chloroformate (30 μL, 0.23 mmol) is added to a cooled (−15° C.) solution of 2-(2-carboxymethoxy-5-chloro-benzyl)-4-chloro-phenoxy]-acetic acid (0.100 g, 0.26 mmol) and TEA (40 μL, 0.26 mmol) in THF (20 mL). After 1.5 hours, the reaction is warmed to 0° C., then MeOH (5 mL) and DMAP (31 mg, 0.25 mmol) are added sequentially and the reaction is stirred at ambient temperature overnight. The solvent is evaporated and the residue is taken into EtOAc containing 10% MeOH, washed with 1 M aqueous HCl and brine, dried (MgSO₄) and evaporated. The crude product is purified by flash chromatography on silica, eluting with 20:1 CH₂Cl₂:MeOH to afford [4-chloro-2-(5-chloro-2-methoxycarbonylmethoxy-benzyl)-phenoxy]-acetic acid.

¹H NMR (DMSO): δ 3.70 (3H, s), 3.90 (2H, s), 4.72 (2H, s), 4.85 (2H, s), 6.89-7.50 (2H, m), 7.18-7.28 (4H, m), 13.0 (1H br, s).

### Examples 2, 3, 4 and 5

These examples, namely, [4-chloro-2-(5-chloro-2-ethoxycarbonylmethoxy-benzyl)-phenoxy]-acetic acid; [4-chloro-2-(5-chloro-2-isobutoxycarbonylmethoxy-benzyl)-phenoxy]-acetic acid; [4-chloro-2-(5-chloro-2-isopropoxycarbonylmethoxy-benzyl)-phenoxy]-acetic acid; and [4-bromo-2-(5-bromo-2-carboxymethoxy-benzyl)-phenoxy]-acetic acid, are prepared by a similar process as that described for Example 1.

### Example 6

### Preparation of [4-chloro-2-(5-chloro-2-cyclopropylcarbamoylmethoxy-benzyl)-phenoxy]-acetic acid

Isobutyl chloroformate (30 μL, 0.23 mmol) is added to a cooled (−15° C.) solution of 2-(2-carboxymethoxy-5-chloro-benzyl)-4-chloro-phenoxy]-acetic acid (Example 1b; 0.10 g, 0.26 mmol) and TEA (40 μL, 0.29 mmol) in THF (20 mL). The reaction is stirred at −15° C. to −10° C. for 2 hours, prior to sequential addition of aminocyclopropane (90 μL, 0.13 mmol) and DMAP (31 mg, 0.26 mmol). After a further 1.5 hours, the reaction mixture is warmed to ambient temperature and the solvent is evaporated. The residue is taken into EtOAc containing 10% MeOH, washed with 1 M aqueous HCl followed by brine, dried Na₂SO₄ and evaporated. The crude product is purified by flash chromatography on silica (20:1-10:1 CH₂Cl₂:MeOH gradient elution) to afford [4-chloro-2-(5-chloro-2-cyclopropylcarbamoylmethoxy-benzyl)-phenoxy]-acetic acid); MH⁺=424.

### Example 7

### a) Preparation of 4-chloro-1-methoxy-2-(2-methoxy-benzyl)-benzene

1-Bromomethyl-2-methoxy-benzene (1.0 g, 5.0 mmol), 5-chloro-2-methoxyphenyl boronic acid (1.03 g, 5.5 mmol) and Pd(PPh₃)₄ (0.311 g, 0.27 mmol) are dissolved in THF (90 mL). A solution of Na₂CO₃ (1.5 g, 14 mmol) in water (4.5 mL) is added and the reaction is heated to reflux with vigorous stirring for 16 hours. The reaction mixture is cooled to room temperature and filtered. The filtrate is evaporated and the residue purified by flash chromatography over silica gel eluting with 5:95 EtOAc:isohexane to afford 4-chloro-1-methoxy-2-(2-methoxy-benzyl)-benzene.

¹H NMR (DMSO-d₆): δ 3.72 (3H, s), 3.74 (3H, s), 3.79 (2H, s), 6.88 (2H, m), 6.97 (3H, m), 7.25 (2H, m).

### b) Preparation of 4-chloro-2-(hydroxyl-benzyl)-phenol

4-Chloro-1-methoxy-2-(2-methoxy-benzyl)-benzene (0.880 g, 3.35 mmol) is dissolved in CH₂Cl₂ (10 mL) and cooled to 0° C. with stirring. Boron tribromide (1 M solution in CH₂Cl₂; 6.7 mL, 6.7 mmol) is added dropwise, maintaining internal temperature <5° C. The solution is stirred at 0° C. for 1 hour and then at ambient temperature for 16 hours. The reaction mixture was poured into water and the organic layer is separated and evaporated. The residue is purified by flash chromatography on silica gel eluting with 15:85 EtOAc:isohexane yielding 4-chloro-2-(hydroxyl-benzyl)-phenol.

¹H NMR (DMSO-d₆): δ 3.75 (2H, s), 6.70 (1H, t), 6.80 (2H, m), 6.85 (1H, d), 6.97 (1H, d), 7.03 (2H, m), 9.50 (2H, br, s).

### c) Preparation of [2-(5-chloro-2-methoxycarbonylmethoxy-benzyl)-phenoxy]-acetic acid methyl ester

4-Chloro-2-(hydroxyl-benzyl)-phenol (0.180 g, 0.77 mmol) and potassium carbonate (0.212 g, 1.53 mmol) are suspended in DMF (2.5 mL). Methyl bromoacetate (0.145 mL, 1.53 mmol) is added and the reaction mixture stirred at room temperature for 16 hours. The reaction mixture is evaporated and water and EtOAc are added to the residue. The organic layer is separated and washed with brine, dried (MgSO₄) and evaporated to yield [2-(5-chloro-2-methoxycarbonylmethoxy-benzyl)-phenoxy]-acetic acid methyl ester.

¹H NMR (DMSO-d₆): δ 3.70 (6H, s), 3.94 (2H, s), 4.83 (2H, s), 4.86 (2H, s), 6.88 (3H, m), 7.12 (1H, d), 7.19 (3H, m).

### d) Preparation of [2-(2-carboxymethoxy-5-chloro-benzyl)-phenoxy]-acetic acid

[2-(5-Chloro-2-methoxycarbonylmethoxy-benzyl)-phenoxy]-acetic acid methyl ester (0.230 g, 0.6 mmol) is dissolved in warm MeOH (5 mL) and cooled to ambient temperature. 2 M aqueous NaOH (1.2 mL, 2.4 mmol) is added and the reaction stirred at ambient temperature for 3 hours. The reaction mixture is evaporated and residue dissolved in water and slowly acidified to pH 1 with 1 M aqueous HCl. The precipitated solid is filtered, washed with water and dried to yield [2-(2-carboxymethoxy-5-chloro-benzyl)-phenoxy]-acetic acid; MH⁺=351.

### Example 8

This example, namely, [4-fluoro-2-(5-fluoro-2-carboxymethoxy-benzyl)-phenoxy]-acetic acid.

### a) Preparation of (5-fluoro-2-methoxy-phenyl)-methanol

Sodium borohydride (61 mg, 1.62 mmol) is added to a solution of 5-fluoro-2-methoxybenzaldehyde (1.0 g, 6.49 mmol) in MeOH (10 mL). After 1 hour, the reaction is poured in to 1 M aqueous HCl and extracted with DCM. The combined organic phases are dried (Na₂SO₄), evaporated and purified by flash chromatography (0-100% EtOAc-isohexanes gradient elution) to afford (5-fluoro-2-methoxy-phenyl)-methanol.

### b) Preparation of 2-bromomethyl-4-fluoro-1-methoxy-benzene

Phosphorus tribromide (0.312 mL, 3.28 mmol) is added to a solution of (5-fluoro-2-methoxy-phenyl)-methanol (1.03 g, 6.56 mmol) in DCM (10 mL). After 30 minutes, the reaction mixture is poured directly on to a silica gel column and eluted with DCM to afford 2-bromomethyl-4-fluoro-1-methoxy-benzene.

### c) Preparation of 1,1′-methylenebis[5-fluoro-2-methoxy-benzene

Sodium carbonate (1.459 g, 13.8 mmol) is added to a mixture of 2-bromomethyl-4-fluoro-1-methoxy-benzene (1.37 g, 6.25 mmol) and 5-fluoro-2-methoxyphenyl boronic acid (1.1.7 g, 6.28 mmol) in THF (20 mL), followed by tetrakis(triphenylphosphine) palladium (0) (0.288 g, 0.25 mmol). The reaction is heated to reflux under argon for 16 hours and evaporated. The crude product is partitioned between water and EtOAc and the combined organic phases are washed with brine, dried (MgSO₄) and evaporated. Flash chromatography (0-100% EtOAc-isohexanes gradient elution) affords crude 1,1′-methylenebis[5-fluoro-2-methoxy-benzene.

### d) Preparation of 2,2′-methylenebis[4-fluorophenol]

Boron tribromide (1 M DCM solution; 8.4 mL, 4.06 mmol) is added to a cooled (0° C.) solution of 1,1′-methylenebis[5-fluoro-2-methoxy-benzene (1.07 g, 4.06 mmol) in DCM (10 mL). The reaction is stirred at RT for 16 hours and quenched dropwise with water (20 mL). The layers are separated and the organic phase is washed with brine, dried (MgSO₄) and evaporated. Flash chromatography (0-40% EtOc-isohexanes gradient elution) followed by crystallisation from water affords 2,2′-methylenebis[4-fluorophenol].

### e) Preparation of [4-fluoro-2-(5-fluoro-2-methoxycarbonylmethoxy-benzyl)-phenoxy]-acetic acid methyl ester

A mixture of 2,2′-methylenebis[4-fluorophenol]. (0.10 g, 0.42 mmol), 2-methyl bromoacetate (77 μL, 0.85 mmol) and potassium carbonate (0.117 g, 0.85 mmol) in DMF (1 mL) is stirred at RT for 16 hours. The solvent is evaporated and the crude product purified by flash chromatography (0-100% EtOAc in isohexane gradient) to afford [4-fluoro-2-(5-fluoro-2-methoxycarbonylmethoxy-benzyl)-phenoxy]-acetic acid methyl ester.

### f) Preparation of [2-(2-carboxymethoxy-5-fluoro-benzyl)-4-fluoro-phenoxy]-acetic acid

NaOH (2 M aqueous; 1 mL, 2.0 mmol) is added to a solution of [4-fluoro-2-(5-fluoro-2-methoxycarbonylmethoxy-benzyl)-phenoxy]-acetic acid methyl ester (0.117 g, 0.31 mmol) in MeOH (10 mL) and the reaction is stirred at RT for 16 hours. The solvent is evaporated, water (5 mL) is added and the mixture is acidified to pH 1 with concentrated HCl. The resultant solid is collected by filtration and dried in vacuo to afford [2-(2-carboxymethoxy-5-fluoro-benzyl)-4-fluoro-phenoxy]-acetic acid; [M−H]-=351.

### Example 9

### a) Preparation of [4-chloro-2-(5-chloro-2-ethoxycarbonylmethoxy-phenylsulfanyl)-phenoxy]-acetic acid ethyl ester

Cesium carbonate (2.3 g, 6.96 mmol) is added portionwise to a solution of bis(2-hydroxy-5-chlorophenyl)sulfide) (1.0 g, 3.48 mmol) in DMF (10 mL), followed by ethyl bromoacetate (0.772 mL, 6.96 mmol). The reaction is stirred at ambient temperature for 16 hours, then poured into 1 M aqueous HCl (100 mL) with ice bath cooling. The product is extracted with EtOAc, washed with brine and dried (Na₂SO₄). Filtration followed by evaporation to dryness in vacuo affords the crude material which is triturated in 1:8 EtOAc:isohexane (10 mL). Filtration, followed by drying in vacuo at 50° C. gives [4-chloro-2-(5-chloro-2-ethoxycarbonylmethoxy-phenylsulfanyl)-phenoxy]-acetic acid ethyl ester.

¹H NMR: δ 1.30 (6H, t), 4.28 (4H, q), 4.68 (4H, s), 4.86 (4H, s), 6.75 (2H, d), 7.20 (4H, m).

### b) Preparation of {4-chloro-2-[2-carboxymethoxy-5-chloro-phenylsulfanyl]-phenoxy}-acetic acid

[4-Chloro-2-(5-chloro-2-ethoxycarbonylmethoxy-phenylsulfanyl)-phenoxy]-acetic acid ethyl ester (0.50 g, 1.09 mmol) is suspended in MeOH:THF (12 mL+10 mL) at ambient temperature. Four (4) M aqueous NaOH (4 mL, 16 mmol) is added and the reaction mixture is stirred at 60° C. for 30 minutes. The reaction mixture is filtered hot, then evaporated and the residue is dissolved in water, cooled in an ice bath and slowly acidified with concentrated HCl to pH 1-2. The precipitated solid is filtered-off, washed with cold water and dried to yield {4-chloro-2-[2-carboxymethoxy-5-chloro-phenylsulfanyl]-phenoxy}acetic acid; [M−H]-=401.

### Example 10

### a) Preparation of {4-chloro-2-[benzenesulfonyl-5-chloro-2-carboxymethoxy]-phenoxy}-acetic acid ethyl ester

Cesium carbonate (613 mg, 1.88 mmol) is added portionwise to a solution of 4-chloro-2-[(5-chloro-2-hydroxy-phenyl)sulfonyl]phenol (300 mg, 0.94 mmol) in DMF (2.5 mL), followed by ethyl bromoacetate (208 μL, 1.88 mmol). The reaction mixture is stirred at ambient temperature for 16 hours, then poured in to 1 M aqueous HCl (100 mL) with ice bath cooling. The product is extracted with EtOAc, washed with brine, and dried (Na₂SO₄). Filtration, followed by evaporation to dryness in vacuo, gives {4-chloro-2-[benzenesulfonyl-5-chloro-2-carboxymethoxy]-phenoxy}acetic acid ethyl ester; MH⁺=491.

### b) Preparation of {4-chloro-2-[benzenesulfonyl-5-chloro-2-carboxymethoxy]-phenoxy}-acetic acid

{4-Chloro-2-[benzenesulfonyl-5-chloro-2-carboxymethoxy]-phenoxy}-acetic acid ethyl ester (70 mg, 0.14 mmol) is suspended in MeOH (3 mL) at ambient temperature. Four (4) M aqueous NaOH (1 mL, 4 mmol) is added and the reaction is stirred at ambient temperature for 2 hours. The reaction mixture is evaporated in vacuo, cooled in an ice bath and slowly acidified with concentrated HCl to pH 1-2. The precipitated solid is filtered, washed with cold water and dried to yield {4-chloro-2-[benzenesulfonyl-5-chloro-2-carboxymethoxy]-phenoxy}-acetic acid; [M−H]-=435.

### Example 11

### a) Preparation of [4-chloro-2-(5-chloro-2-hydroxy-benzyl)-phenoxy]-acetic acid benzyl ester

2,2′-Methylene-bis(4-chloro-phenol) (12 g, 44.6 mmol) is dissolved in DMF (100 mL). Lithium carbonate (3.3 g, 44.6 mmol) was added, followed by benzyl-2-bromoacetate (7.7 mL, 49 mmol). The suspension is stirred at 80° C. for 8 hours. Further benzyl-2-bromoacetate (1 mL, 6.4 mmol) is added and stirring continued at 100° C. for 4 hours. The reaction mixture is evaporated to dryness, water is added to the residue which is acidified to pH 1 with 2 M aqueous HCl and extracted with EtOAc. The organic layer is washed with brine, dried (MgSO₄) and evaporated. The crude product is purified by flash column chromatography over silica gel eluting with 4:1 isohexane:EtOAc. The product is suspended in isohexanes, dissolved in the minimum volume of EtOAc, seeded and left to stand. The resultant solid is collected by filtration, washed with isohexane and dried to give [4-chloro-2-(5-chloro-2-hydroxy-benzyl)-phenoxy]-acetic acid benzyl ester; m.p.=135-137° C.

### b) Preparation of 4-[2-(2-benzyloxycarbonylmethoxy-5-chloro-benzyl)-4-chloro-phenoxy]-butyric acid methyl ester

A solution comprising [4-chloro-2-(5-chloro-2-hydroxy-benzyl)-phenoxy]-acetic acid benzyl ester (0.21 g, 0.5 mmol), methyl-4-bromo butyrate (0.091 g, 0.5 mmol) and potassium carbonate (0.138 g, 1 mmol) in DMF (2 mL) is stirred at room temperature for 60 hours. The reaction mixture is concentrated in vacuo and water (20 mL) is added to the crude residue. The aqueous portion is extracted with EtOAc (20 mL) and the organic layer is washed with brine (10 mL), dried (MgSO₄) and evaporated to give 4-[2-(2-benzyloxycarbonylmethoxy-5-chloro-benzyl)-4-chloro-phenoxy]-butyric acid methyl ester which is used crude in the next step.

### c) Preparation of 4-[2-(2-carboxymethoxy-5-chloro-benzyl)-4-chloro-phenoxy]-butyric acid

4-[2-(2-Benzyloxycarbonylmethoxy-5-chloro-benzyl)-4-chloro-phenoxy]-butyric acid methyl ester (0.2 g, 0.386 mmol) is dissolved in THF (5 mL) and treated with 2 M NaOH (0.773 mL, 1.55 mmol), while stirring. The reaction mixture is allowed to stir at room temperature overnight. The solvent is removed in vacuo and the residue is dissolved in water (10 mL) and acidified using 2 M HCl (1 mL), while stirring. The resultant precipitate is filtered, washed with water and dried under vacuum to afford 4-[2-(2-carboxymethoxy-5-chloro-benzyl)-4-chloro-phenoxy]-butyric acid as a white solid; MH⁺=413.

### Example 12

### a) Preparation of 4-chloro-1-methoxy-2-(2-methoxy-benzyl)-nitrobenzene

2-Methoxy-5-nitrobenzyl bromide (3.23 g, 13.11 mmol) and 5-chloro-2-methoxyphenyl boronic acid (2.43 g, 13.11 mmol) are dissolved in THF (180 mL) and treated with Pd(PPh₃)₄ (0.72 g, 0.625 mmol). The reaction mixture is stirred under an atmosphere of argon in the dark and a solution of Na₂CO₃ (3.5 g, 33 mmol) in water (10 mL) is added and the reaction is heated to reflux for 23 hours. The reaction mixture is cooled to room temperature and filtered. The filtrate is evaporated and the residue purified by flash chromatography over silica gel eluting with 25:75 EtOAc:isohexane to afford 4-chloro-1-methoxy-2-(2-methoxy-benzyl)-nitrobenzene; MH⁺=308.

### b) Preparation of 4-chloro-2-(2-hydroxy-nitrobenzyl)-phenol

A solution comprising 4-chloro-1-methoxy-2-(2-methoxy-benzyl)-nitrobenzene (1.5 g, 4.87 mmol) in toluene (15 mL) is treated with beryllium chloride (2.3 g, 28.8 mmol). The reaction mixture is stirred at reflux under an atmosphere of argon for 23 hours and then cooled to room temperature. The solvent is removed in vacuo and the crude residue is taken up in an aqueous solution of 2 M HCl. The solution is extracted with CHCl₃ (150 mL) and the organic portion is dried (MgSO₄) and evaporated to dryness. The residue is purified by flash chromatography over silica gel eluting with 20:80 EtOAc:isohexane to afford 4-chloro-2-(2-hydroxy-nitrobenzyl)-phenol.

### c) Preparation of [2-(5-chloro-2-methoxycarbonylmethoxy-benzyl)-4-nitro-phenoxy]-acetic acid methyl ester

4-Chloro-2-(2-hydroxy-nitrobenzyl)-phenol (0.091 g, 0.33 mmol) and potassium carbonate (0.0.91 g, 0.66 mmol) are suspended in DMF (3 mL). Methyl bromoacetate (0.063 mL, 0.66 mmol) is added and the reaction mixture stirred at room temperature for 17 hours. The reaction mixture is evaporated and EtOAc are added to the residue. The organic portion is washed with brine, dried (MgSO₄) and evaporated to dryness to afford [2-(5-chloro-2-methoxycarbonylmethoxy-benzyl)-4-nitro-phenoxy]-acetic acid methyl ester.

### d) Preparation of [2-(2-carboxymethoxy-5-chloro-benzyl)-4-nitro-phenoxy]-acetic acid

[2-(5-Chloro-2-methoxycarbonylmethoxy-benzyl)-4-nitro-phenoxy]-acetic acid methyl ester (0.076 g, 0.18 mmol) is dissolved in warm MeOH (5 mL) and treated with 2 M aqueous NaOH (0.36 mL, 0.72 mmol). A few drops of THF is added to aid dissolution. The reaction mixture is cooled to ambient temperature and stirred for 2.5 hours. The reaction mixture is evaporated and the residue dissolved in water and slowly acidified to pH 1 with 1 M aqueous HCl. The precipitated solid is filtered, washed with water and dried to yield [2-(2-carboxymethoxy-5-chloro-benzyl)-4-nitro-phenoxy]-acetic acid; [M−H]-=394.

### Example 13

### a) Preparation of (4-chloro-2-(5-chloro-2-hydroxy-benzyl)-phenoxy]-acetic acid benzyl ester

2,2′-Methylene-bis(4-chloro-phenol) (12 g, 44.6 mmol) is dissolved in DMF (100 mL). Lithium carbonate (3.3 g, 44.6 mmol) was added, followed by benzyl-2-bromoacetate (7.7 mL, 49 mmol). The suspension is stirred at 80° C. for 8 hours. Further benzyl-2-bromoacetate (1 mL, 6.4 mmol) is added and stirring continued at 100° C. for 4 hours. The reaction mixture is evaporated to dryness, water is added to the residue which is acidified to pH 1 with 2 M aqueous HCl and extracted with EtOAc. The organic layer is washed with brine, dried (MgSO₄) and evaporated. The crude product is purified by flash column chromatography over silica gel eluting with 4:1 isohexane:EtOAc. The product is suspended in isohexanes, dissolved in the minimum volume of EtOAc, seeded and left to stand. The resultant solid is collected by filtration, washed with isohexane and dried to give [4-chloro-2-(5-chloro-2-hydroxy-benzyl)-phenoxy]-acetic acid benzyl ester; m.p.=135-137° C.

### b) Preparation of 2-[2-(2-carboxymethoxy-5-chloro-benzyl)-4-chloro-phenoxy]-2-methyl-propionic acid

[4-Chloro-2-(5-chloro-2-hydroxy-benzyl)-phenoxy]-acetic acid benzyl ester (0.5 g, 1.20 mmol), CHCl₃ (0.13 mL, 1.62 mmol), NaOH pellets (0.268 g, 6.71 mmol) are added to acetone (1.43 mL) and sonicated to aid dissolution. The reaction mixture is heated to reflux for 7 hours and further portions of acetone are added gradually (1.5 mL in total) during heating. The solvent is removed in vacuo and the residue is taken up in water. The pH of the aqueous portion is adjusted to pH 12 with 2 M NaOH and then washed with DCM and EtOAc. The solution is then acidified to pH 0 with 2 M HCl. The resulting precipitate is extracted with EtOAc and the organic layer is dried (MgSO₄) and evaporated. The crude residue is purified by mass directed preparative HPLC to yield the titled compound; [M−H]⁻=411.

### Example 14

### a) Preparation of (4-chloro-2-formyl-phenoxy)-acetic acid methyl ester

5-Chloro-2-hydroxy-benzaldehyde (3.9 g, 22.7 mmol) is dissolved in DMF (20 mL) and potassium carbonate (4.7 g, 34.1 mmol) is added. After stirring the suspension for 10 minutes at ambient temperature, methyl bromoacetate (3.22 mL, 34.1 mmol) is added. The suspension is stirred at ambient temperature for 16 hours. The reaction mixture is evaporated to dryness. Water is added to the residue and the mixture extracted with EtOAc. The organic layer is washed with water and brine. The organic layer is dried (MgSO₄) and evaporated to dryness to give (4-chloro-2-formyl-phenoxy)-acetic acid methyl ester.

¹H NMR (DMSO-d₆): δ 3.70 (3H, s), 5.05 (2H, s), 7.27 (1H, d), 7.65 (1H, d), 7.70 (1H, dd), 10.35 (1H, s).

### b) Preparation of (4-chloro-2-hydroxymethyl-phenoxy)-acetic acid methyl ester

(4-Chloro-2-formyl-phenoxy)-acetic acid methyl ester (5 g, 22 mmol) is dissolved in methanol under argon atmosphere and cooled to 0° C. under stirring. Sodium borohydride (0.413 g, 11 mmol) is added and the reaction mixture stirred at 0° C. for 1 hour. Water (1 mL) is added and the reaction mixture evaporated to dryness. EtOAc is added to the residue and the mixture washed with water and brine. The organic layer is dried (MgSO₄) and evaporated to dryness. The residue is purified by flash chromatography using 1:3 EtOAc:isohexane as eluent to give (4-chloro-2-hydroxymethyl-phenoxy)-acetic acid methyl ester.

¹H NMR (DMSO-d₆): δ 1.20 (3H, t), 4.15 (2H, q), 4.55 (2H, d), 4.82 (2H, s), 5.25 (1H, t), 6.90 (1H, d), 7.22 (1H, dd), 7.38 (1H, s).

### c) Preparation of (2-bromomethyl-4-chloro-phenoxy)-acetic acid methyl ester

(4-Chloro-2-hydroxymethyl-phenoxy)-acetic acid ethyl ester (0.500 g, 2.04 mmol) is dissolved in DCM (10 mL) and cooled to 0° C. with stirring. Phosphorus tribromide (0.116 mL, 1.02 mmol) is added and the reaction mixture stirred at 0° C. for 30 minutes. The reaction mixture is purified by flash chromatography eluting with DCM to afford (2-bromomethyl-4-chloro-phenoxy)-acetic acid ethyl ester.

¹H NMR (DMSO-d₆): δ 1.20 (3H, t), 4.15 (2H, q), 4.65 (2H, s), 4.91 (2H, s), 7.00 (1H, d), 7.35 (1H, dd), 7.55 (1H, d).

### d) Preparation of (E)-3-[2-(2-carboxymethoxy-5-chloro-benzyl)-phenyl]-acrylic acid

(2-Bromomethyl-4-chloro-phenoxy)-acetic acid methyl ester (0.7 g, 2.38 mmol) and 2-2-carboxyvinylbenzene boronic acid (0.5 g, 2.62 mmol) are dissolved in THF (33 mL) under an inert atmosphere of argon. The stirred reaction mixture is then treated with Pd(PPh₃)₄ (0.131 g, 0.14 mmol) and a solution of Na₂CO₃ (0.64 g, 5.99 mmol) in water (2 mL) is added and the reaction is heated to reflux with vigorous stirring for 21 hours. The reaction mixture is cooled to room temperature and filtered. The filtrate is evaporated and the residue purified by flash chromatography over silica gel eluting with MeOH:DCM (1:4 increasing to 4:1) to afford (E)-3-[2-(2-carboxymethoxy-5-chloro-benzyl)-phenyl]-acrylic acid; [M−H]-=345.

### e) Preparation of 3-[2-(2-carboxymethoxy-5-chloro-benzyl)-phenyl]-propionic acid

(E)-3-[2-(2-Carboxymethoxy-5-chloro-benzyl)-phenyl]-acrylic acid (0.122 g, 0.35 mmol) is dissolved in MeOH (10 mL) and stirred at room temperature under an atmosphere of argon. The solution is then treated with 5% palladium on carbon (0.018 g). The reaction mixture is purged with argon and then twice with nitrogen and placed under an atmosphere of hydrogen for 5 hours. The mixture is then filtered through Celite™ filter material and the solvent is removed in vacuo to give 3-[2-(2-carboxymethoxy-5-chloro-benzyl)-phenyl]-propionic acid; [M−H]-=347.

### Example 15

### a) Preparation of [2-(2-benzyloxycarbonylmethoxy-5-chloro-benzyl)-4-chlorophenoxy]-acetic acid methyl ester

[4-Chloro-2-(5-chloro-2-hydroxy-benzyl)-phenoxy]-acetic acid benzyl ester (3.53 g, 8.46 mmol), methyl bromoacetate (0.803 mL, 8.46 mmol), potassium carbonate (1.17 g, 8.46 mmol) and tetrabutylammonium iodide is suspended in DMF (20 mL) and stirred at ambient temperature for 16 hours. The reaction mixture is evaporated to dryness. EtOAc is added to the residue and the mixture washed with 1 M aqueous HCl and brine, dried (MgSO₄) and evaporated to yield [2-(2-tert-butoxycarbonylmethoxy-5-chlorobenzyl)-4-chloro-phenoxy]-acetic acid benzylester; [M+H]⁺=456?.

### b) Preparation of [2-(2-benzyloxycarbonylmethoxy-5-chloro-benzoyl)-4-chloro-phenoxy]-acetic acid methyl ester

A solution of chromium trioxide (1.07 g, 10.71 mmol) in acetic acid (14 mL) is added portionwise to a hot (60° C.) solution of [2-(2-benzyloxycarbonylmethoxy-5-chloro-benzyl)-4-chlorophenoxy]-acetic acid methyl ester (0.906 g, 1.85 mmol) in acetic acid (7 mL). After 5 minutes the reaction is cooled to room temperature, diluted with water and extracted with ether. The organic phase is dried (MgSO₄), evaporated and purified by flash chromatography over silica gel, eluting with 4:1 isohexane:EtOAc to afford [2-(2-benzyloxycarbonylmethoxy-5-chloro-benzoyl)-4-chloro-phenoxy]-acetic acid methyl ester; MH⁺=503.

### c) Preparation of [2-(2-carboxymethoxy-5-chloro-benzoyl)-4-chloro-phenoxy]-acetic acid

Two (2) M aqueous NaOH (0.70 mL, 1.59 mmol) is added to a solution of [2-(2-benzyloxycarbonylmethoxy-5-chloro-benzoyl)-4-chloro-phenoxy]-acetic acid methyl ester (0.200 g, 0.397 mmol) in MeOH (5 mL). The reaction is stirred for 1 hour at RT and the solvent is evaporated. The residue is partitioned between water and ether and the aqueous phase is acidified to pH 1 with 2 M HCl. After extraction with CH₂Cl₂, the organic phase is dried (MgSO₄) and evaporated to afford [2-(2-carboxymethoxy-5-chloro-benzoyl)-4-chloro-phenoxy]-acetic acid; [M−H]⁻=397.

### Example 16

### a) Preparation of [4-chloro-2-(2-hydroxy-5-methanesulfonyl-benzyl)-phenoxy]-acetic acid methyl ester

(4-Chloro-2-hydroxymethyl-phenoxy)-acetic acid methyl ester (230 mg, 1.0 mmol) and 4-methylsulfonyl phenol (0.340 g, 2.0 mmol) are melted in a microwave reaction vessel and zinc chloride (0.680 g, 5.0 mmol) is added. The mixture is heated in the Emrys Optimizer™ microwave for 2 minutes at 180° C. Warm water is added and the mixture is extracted with EtOAc. The organic layer is evaporated and the residue purified by reversed phase flash chromatography over C18 silica, eluting with a gradient of acetonitrile:water 0:100 to 100:0 over 30 minutes to afford [4-chloro-2-(2-hydroxy-5-methanesulfonyl-benzyl)-phenoxy]-acetic acid methyl ester; MH⁺=385.

### b) Preparation of [2-(2-tert-butoxycarbonylmethoxy-5-methanesulfonyl-benzyl)-4-chloro-phenoxy]-acetic acid methyl ester

[4-Chloro-2-(2-hydroxy-5-methanesulfonyl-benzyl)-phenoxy]-acetic acid methyl ester (60 mg, 0.16 mmol) is dissolved in DMF (1 mL) and potassium carbonate (32 mg, 0.23 mmol) is added. After stirring for 10 minutes at room temperature, tert-butyl-2-bromo acetate (46 mg, 0.23 mmol) is added and the suspension stirred for 16 hours at RT. The reaction mixture is evaporated. The residue is partitioned between water and EtOAc and the organic phase is washed with brine. The organic phase is dried (MgSO₄) and evaporated. The residue is purified by flash chromatography using EtOAc:isohexane 1:1 as eluent to afford [2-(2-tert-butoxycarbonylmethoxy-5-methanesulfonyl-benzyl)-4-chloro-phenoxy]-acetic acid methyl ester; [MH⁺−tBu]=443.

### c) Preparation of [2-(2-carboxymethoxy-5-methanesulfonyl-benzyl)-4-chloro-phenoxy]-acetic acid

[2-(2-tert-Butoxycarbonylmethoxy-5-methanesulfonyl-benzyl)-4-chloro-phenoxy]-acetic acid methyl ester (60 mg, 0.12 mmol) is dissolved in THF (1 mL) and 2 M NaOH (0.12 mL, 0.24 mmol) is added. After stirring for 3 hours at RT, 2 M HCl (0.12 mL) is added and the reaction mixture is evaporated to dryness. The residue is partitioned between water and EtOAc. The organic phase is dried (MgSO₄) and evaporated. The residue is triturated with CH₂Cl₂ to afford [2-(2-carboxymethoxy-5-methanesulfonyl-benzyl)-4-chloro-phenoxy]-acetic acid; MH⁺=429.

The following examples have been prepared using the process described above in the Examples.

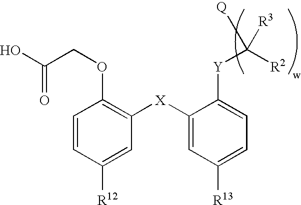

| Example | R¹² | R¹³ | Q | MH⁺ |
|---|---|---|---|---|
| 1 | Cl | Cl | 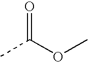 | See NMR data |
| 2 | Cl | Cl | 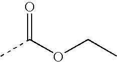 | 413 |
| 3 | Cl | Cl | 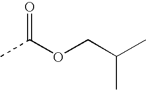 | 441 |
| 4 | Cl | Cl | 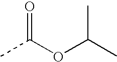 | 427 |
| 5 | Br | Br | 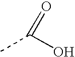 | 473 |
| 6 | Cl | Cl | 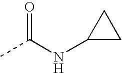 | 424 |
| 7 | H | Cl | 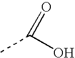 | 351 |
| 8 | F | F | 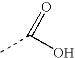 | 353 |
| 9 | Cl | Cl | 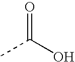 | [M − H]⁻ 401 |
| 10 | Cl | Cl | 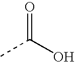 | 435 |
| 11 | Cl | Cl | 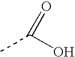 | 413 |
| 12 | NO₂ | Cl | 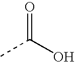 | [M − H]⁻ 394 |
| 13 | Cl | Cl | 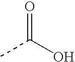 | [M − H])⁻ 411 |
| 14 | Cl | H | 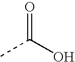 | [M − H]⁻ 347 |
| 15 | Cl | Cl | 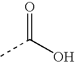 | [M − H]⁻ 397 |
| 16 | Cl | SO₂CH₃ | 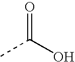 | 429 |

MH⁺ refers to monoisotopic molecular weights
R² = R³ = H
X = CH₂
Y = O
w = 1 except where indicated

### Pharmaceutical Use and Assay

Compounds of formulae (I) and (Ia) and their pharmaceutically acceptable salts, hereinafter referred to alternatively as “agents of the invention”, are useful as pharmaceuticals. In particular, the compounds have good CRTh₂ receptor antagonist activity and may be tested in the following assays.

Scintillation Proximity Assay (SPA) protocol

Membranes are prepared from K562 or Chinese Hamster Ovary (CHO) cells stably transfected with human CRTh₂ receptors.

The assay is performed in a 96-well U-bottomed polypropylene plate in a final volume of 100 μL and the components of the assay are added as follows: test compound in DMSO/assay buffer (25 μL), ³H prostaglandin D₂ (PGD₂) (25 μL) and CRTh₂ membrane fragments (50 μL). The assay is incubated at ambient temperature with shaking for 60 minutes, then harvested on to filter plates. The plate is dried for 2 hours, prior to addition of Micro-Scint 20M (50 μL) and sealing with TopSeal-S™. Plates are then counted using a Packard Top Count instrument, each well being counted for 20 minutes. Ki values are determined using Sigma Plot™ software.

CRTh₂ cAMP Assay Protocol

Test compounds are prepared in assay stimulation buffer/DMSO and 5 μL/well is added to an assay plate (384-well white opti-plate).

CHO cells stably transfected with the CRTh₂ receptor are prepared (dissociated from a cell culture flask and washed in PBS) to a concentration of 4×10⁶/mL in assay stimulation buffer and added to the assay plate (10 μL/well).

The assay plate is incubated at room temperature on a shaker for 15 minutes.

A mix of agonist (10 nM PGD₂) and 5 μM forskolin is prepared in assay stimulation buffer and added to the assay plate (5 μL/well).

In addition, a cAMP standard is serially diluted in assay stimulation buffer and added to separate empty wells on the assay plate (20 μL/well).

The assay plate is incubated at room temperature on a shaker for 60 minutes.

A cell lysis mix (lysis buffer containing Alphascreen™ donor beads and biotinylated cAMP) is prepared under darkened conditions 60 minutes prior to addition. Alphascreen™ acceptor beads are added to the lysis mix after 60 minutes. The resulting lysis mix is added to all wells of the assay plate (40 μL/well).

The assay plate is sealed with Topseal-S™ and incubated in the dark at room temperature on a shaker for 45 minutes. The plate is then counted using a Packard Fusion™ instrument.

The resulting counts per minute are converted to nM cAMP by using the prepared cAMP standard curve. IC₅₀ values are then determined using Prism™ software.

Compounds of the Examples, herein, generally have Ki values in the SPA binding assay below 1 μM. The compounds also generally have IC₅₀ values in the functional assays below 1 μM.

Compounds of the Examples, herein below, generally have Ki values in the SPA binding assay below 1 μM. For example, the compounds of Examples 2 and 13 have Ki values of 0.0.008 and 0.058 μM, respectively.

Compounds of the Examples, herein below, generally have IC₅₀ values in the functional assay below 1 μM. For example, the compounds of Examples 2, and 13 have IC₅₀ values of 0.068 and 0.052 μM, respectively.

Compounds of formulae (I) and (Ia), in free or salt form, are antagonists of the G-protein-coupled chemoattractant receptor CRTh₂, expressed on Th₂ cells, eosinophils and basophils. PGD₂ is the natural ligand for CRTh₂. Thus, antagonists which inhibit the binding of CRTh₂ and PGD₂ are useful in the treatment of allergic and anti-inflammatory conditions. Treatment in accordance with the invention may be symptomatic or prophylactic.

Accordingly, agents of the invention are useful in the treatment of inflammatory or obstructive airways diseases, resulting, e.g., in reduction of tissue damage, airways inflammation, bronchial hyperreactivity, remodeling or disease progression. Inflammatory or obstructive airways diseases to which the present invention is applicable include asthma of whatever type or genesis including both intrinsic (non-allergic) asthma and extrinsic (allergic) asthma, mild asthma, moderate asthma, severe asthma, bronchitis asthma, exercise-induced asthma, occupational asthma and asthma induced following bacterial infection. Treatment of asthma is also to be understood as embracing treatment of subjects, e.g., of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g., of acute asthmatic or bronchoconstrictor attack, improvement in lung function or improved airways hyperreactivity. It may further be evidenced by reduced requirement for other, symptomatic therapy, i.e., therapy for or intended to restrict or abort symptomatic attack when it occurs, e.g., anti-inflammatory (e.g., corticosteroid) or bronchodilatory. Prophylactic benefit in asthma may, in particular, be apparent in subjects prone to “morning dipping”. “Morning dipping” is a recognized asthmatic syndrome, common to a substantial percentage of asthmatics and characterized by asthma attack, e.g., between the hours of about 4-6 a.m., i.e., at a time normally substantially distant from any previously administered symptomatic asthma therapy.

Other inflammatory or obstructive airways diseases and conditions to which the present invention is applicable include acute lung injury (ALI), adult respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular, other inhaled drug therapy. The invention is also applicable to the treatment of bronchitis of whatever type or genesis including, e.g., acute, arachidic, catarrhal, croupus, chronic or phthinoid bronchitis. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis including, e.g., aluminosis, anthracosis, asbestosis, chalicosis, ptilosis, siderosis, silicosis, tabacosis and byssinosis.

Having regard to their anti-inflammatory activity, in particular, in relation to inhibition of eosinophil activation, agents of the invention are also useful in the treatment of eosinophil related disorders, e.g., eosinophilia, in particular, eosinophils-related disorders of the airways, e.g., involving morbid eosinophilic infiltration of pulmonary tissues including hypereosinophilia as it effects the airways and/or lungs, as well as, e.g., eosinophil-related disorders of the airways consequential or concomitant to Loffler's syndrome; eosinophilic pneumonia; parasitic, in particular, metazoan, infestation including tropical eosinophilia; bronchopulmonary aspergillosis; polyarteritis nodosa including Churg-Strauss syndrome; eosinophilic granuloma; and eosinophil-related disorders affecting the airways occasioned by drug-reaction.

Agents of the invention are also useful in the treatment of inflammatory or allergic conditions of the skin, e.g., psoriasis, contact dermatitis, atopic dermatitis, alopecia greata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita and other inflammatory or allergic conditions of the skin.

Agents of the invention may also be used for the treatment of other diseases or conditions, in particular, diseases or conditions having an inflammatory component, e.g., treatment of diseases and conditions of the eye, such as conjunctivitis, keratoconjunctivitis sicca and vernal conjunctivitis; diseases affecting the nose including allergic rhinitis; and inflammatory disease, in which autoimmune reactions are implicated or having an autoimmune component or aetiology, including autoimmune hematological disorders, e.g., hemolytic anemia, aplastic anaemia, pure red cell anaemia and idiopathic thrombocytopenia; systemic lupus erythematosus; polychondritis; scierodoma; Wegener granulamatosis; dermatomyositis; chronic active hepatitis; myasthenia gravis; Steven-Johnson syndrome; idiopathic sprue; autoimmune inflammatory bowel disease, e.g., ulcerative colitis and Crohn's disease; endocrine opthalmopathy; Grave's disease; sarcoidosis; alveolitis; chronic hypersensitivity pneumonitis; multiple sclerosis; primary billiary cirrhosis; uveitis (anterior and posterior); keratoconjunctivitis sicca and vernal keratoconjunctivitis; interstitial lung fibrosis; psoriatic arthritis; and glomerulonephritis, with and without nephrotic syndrome, e.g., including idiopathic nephrotic syndrome or minal change nephropathy.

Other diseases or conditions which may be treated with agents of the invention include septic shock; rheumatoid arthritis; osteoarthritis; proliferative diseases, such as cancer; atherosclerosis; allograft rejection following transplantation; stroke; obesity; restenosis; diabetes, e.g., diabetes mellitus type I (juvenile diabetes) and diabetes mellitus type II; diarrheal diseases; ischemia/reperfusion injuries; retinopathy, such as diabetic retinopathy or hyperbaric oxygen-induced retinopathy; and conditions characterized by elevated intraocular pressure or secretion of ocular aqueous humor, such as glaucoma.

The effectiveness of an agent of the invention in inhibiting inflammatory conditions, e.g., in inflammatory airways diseases, may be demonstrated in an animal model, e.g., a mouse or rat model, of airways inflammation or other inflammatory conditions, e.g., as described by Szarka et al., J Immunol Methods, Vol. 202, pp. 49-57 (1997); Renzi et al., Am Rev Respir Dis, Vol. 148, pp. 932-939 (1993); Tsuyuki et al., J Clin Invest, Vol. 96, pp. 2924-2931 (1995); Cernadas et al., Am J Respir Cell Mol Biol, Vol. 20, pp. 1-8 (1999); and Williams and Galli, J Exp Med, Vol. 192, pp. 455-462 (2000).

The agents of the invention are also useful as co-therapeutic agents for use in combination with other drug substances, such as anti-inflammatory, bronchodilatory or antihistamine drug substances, particularly in the treatment of obstructive or inflammatory airways diseases, such as those mentioned hereinbefore, e.g., as potentiators of therapeutic activity of such drugs or as a means of reducing required dosaging or potential side effects of such drugs. An agent of the invention may be mixed with the other drug substance in a fixed pharmaceutical composition or it may be administered separately, before, simultaneously with or after the other drug substance. Accordingly the invention includes a combination of an agent of the invention as hereinbefore described with an anti-inflammatory, bronchodilatory, antihistamine or anti-tussive drug substance, said agent of the invention and said drug substance being in the same or different pharmaceutical composition.

Such anti-inflammatory drugs include steroids, in particular, glucocorticosteroids, such as budesonide, beclamethasone dipropionate, fluticasone propionate, ciclesonide or mometasone furoate; or steroids, described in WO 02/88167, WO 02/12266, WO 02/100879, WO 02/00679 (especially those of Examples 3, 11, 14, 17, 19, 26, 34, 37, 39, 51, 60, 67, 72, 73, 90, 99 and 101), WO 03/035668, WO 03/048181, WO 03/062259, WO 03/064445 and WO 03/072592, WO 04/039827, WO 04/066920; non-steroidal glucocorticoid receptor agonists, such as those described in WO 00/00531, WO 02/10143, DE 10261874, WO 03/082280, WO 03/082787, WO 03/104195, WO 03/101932, WO 04/019935, WO 04/018429, WO 04/005229, WO 03/086294 and WO 04/26248; LTB4 antagonists, such as those described in U.S. Pat. No. 5,451,700; LTD4 antagonists, such as montelukast and zafirlukast; PDE4 inhibitors, such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659 (Parke-Davis), AWD-12-281 (Asta Medica), CDC-801 (Celgene), SeICID™ CC-10004 (Celgene), KW4490 (Kyowa Hakko Kogyo), WO 03/104204, WO 03/104205, WO 04/000814, WO 04/000839 and WO 04/005258 (Merck), as well as those described in WO 98/18796 and WO 03/39544; A2a agonists, such as those described in EP 1052264, EP 1241176, EP 409595A2, WO 94/17090, WO 96/02543, WO 96/02553, WO 98/28319, WO 99/24449, WO 99/24450, WO 99/24451, WO 99/38877, WO 99/41267, WO 99/67263, WO 99/67264, WO 99/67265, WO 99/67266, WO 00/23457, WO 00/77018, WO 00/78774, WO 01/23399, WO 01/27130, WO 01/27131, WO 01/60835, WO 01/94368, WO 02/00676, WO 02/22630, WO 02/96462 and WO 03/086408; A2b antagonists, such as those described in WO 02/42298; and beta (β)-2-adrenoceptor agonists, such as albuterol (salbutamol), metaproterenol, terbutaline, salmeterol, fenoterol, procaterol, and especially, formoterol and pharmaceutically acceptable salts thereof, and compounds (in free or salt or solvate form) of formula (I) of WO 00/75114, which document is incorporated herein by reference, preferably compounds of the Examples thereof, especially a compound of formula

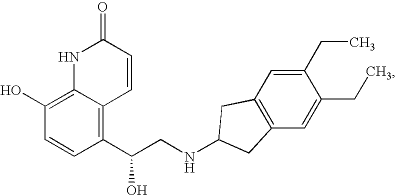

and pharmaceutically acceptable salts thereof, as well as compounds (in free or salt or solvate form) of formula (I) of WO 04/16601. Further β-2-adrenoreceptor agonists include compounds of JP 05025045, WO 93/18007, WO 99/64035, U.S. Patent No. 2002/0055651, WO 01/42193, WO 01/83462, WO 02/66422, WO 02/70490, WO 02/76933, WO 03/024439, WO 03/072539, WO 03/042160, WO 03/091204, WO 03/042164, WO 03/099764, WO 04/016578, WO 04/022547, WO 04/032921, WO 04/037773, WO 04/037807, WO 04/039762, WO 04/039766, WO 04/045618, WO 04/046083, WO 04/033412, WO 04/037768, WO 04/037773 and EP 1440966.

Such bronchodilatory drugs include anticholinergic or antimuscarinic agents, in particular, ipratropium bromide, oxitropium bromide, tiotropium salts and CHF 4226 (Chiesi), but also those described in WO 01/04118, WO 02/51841, WO 02/53564, WO 03/00840, WO 03/87094, WO 04/05285, WO 02/00652, WO 03/53966, EP 0424021, U.S. Pat. No. 5,171,744, U.S. Pat. No. 3,714,357 and WO 03/33495.

Such co-therapeutic antihistamine drug substances include cetirizine hydrochloride, acetaminophen, clemastine fumarate, promethazine, loratidine, desloratidine, diphenhydramine and fexofenadine hydrochloride.

Combinations of agents of the invention and steroids, β-2 agonists, PDE4 inhibitors or LTD4 antagonists may be used, e.g., in the treatment of COPD or, particularly, asthma. Combinations of agents of the invention and anticholinergic or antimuscarinic agents, PDE4 inhibitors, dopamine receptor agonists or LTB4 antagonists may be used, e.g., in the treatment of asthma or, particularly, COPD.

Other useful combinations of agents of the invention with anti-inflammatory drugs are those with antagonists of chemokine receptors, e.g., CCR-1, CCR-2, CCR-3, CCR-4, CCR-5, CCR-6, CCR-7, CCR-8, CCR-9, CCR-10, CXCR1, CXCR2, CXCR3, CXCR4 and CXCR5; particularly useful are CCR-3 antagonists, such as those described in WO 02/026723, especially 4-{3-[(S)-4-(3,4-dichlorobenzyl)-morpholin-2-ylmethyl]-ureidomethyl}-benzamide and those described in WO 03/077907, WO 03/007939 and WO 02/102775.

Also especially useful are CCR-5 antagonists, such as Schering-Plough antagonists SC-351125, SCH-55700 and SCH-D; Takeda antagonists, such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770); and CCR-5 antagonists, described in U.S. Pat. No. 6,166,037, WO 00/66558 and WO 00/66559.

The agents of the invention may be administered by any appropriate route, e.g., orally, e.g., in the form of a tablet or capsule; parenterally, e.g., intravenously; by inhalation, e.g., in the treatment of inflammatory or obstructive airways disease; intranasally, e.g., in the treatment of allergic rhinitis; topically to the skin, e.g., in the treatment of atopic dermatitis; or rectally, e.g., in the treatment of inflammatory bowel disease.

The present invention also provides a pharmaceutical composition comprising a compound of formula (I), in free form or in the form of a pharmaceutically acceptable salt, optionally together with a pharmaceutically acceptable diluent or carrier therefore. The composition may contain a co-therapeutic agent, such as an anti-inflammatory, bronchodilatory or antihistamine drug, as hereinbefore described. Such compositions may be prepared using conventional diluents or excipients and techniques known in the galenic art. Thus oral dosage forms may include tablets and capsules. Formulations for topical administration may take the form of creams, ointments, gels or transdermal delivery systems, e.g., patches. Compositions for inhalation may comprise aerosol or other atomizable formulations or dry powder formulations.

The present invention also provides for the use of a compound of the present invention in any of the aforementioned embodiments, in free or pharmaceutically acceptable salt form, for the manufacture of a medicament for the treatment of an inflammatory or allergic condition, particularly an inflammatory or obstructive airways disease.

The present invention also provides a method for treating or preventing inflammatory or allergic conditions comprising administering to a patient in need thereof a therapeutically effective amount of a compound of the present invention, in free or a pharmaceutically acceptable salt form.

When the composition comprises an aerosol formulation, it preferably contains, e.g., a hydro-fluoro-alkane (HFA) propellant, such as HFA134a or HFA227 or a mixture of these, and may contain one or more co-solvents known in the art, such as ethanol (up to 20% by weight); and/or one or more surfactants, such as oleic acid or sorbitan trioleate; and/or one or more bulking agents, such as lactose. When the composition comprises a dry powder formulation, it preferably contains, e.g., the compound of formula (I) having a particle diameter up to 10 microns, optionally together with a diluent or carrier, such as lactose, of the desired particle size distribution and a compound that helps to protect against product performance deterioration due to moisture. When the composition comprises a nebulized formulation, it preferably contains, e.g., the compound of formula (I), either dissolved or suspended, in a vehicle containing water, a co-solvent, such as ethanol or propylene glycol and a stabilizer, which may be a surfactant.

The invention includes:
(a) an agent of the invention in inhalable form, e.g., in an aerosol or other atomizable composition or in inhalable particulate, e.g., micronized form;
(b) an inhalable medicament comprising an agent of the invention in inhalable form;
(c) a pharmaceutical product comprising such an agent of the invention in inhalable form in association with an inhalation device; and
(d) an inhalation device containing an agent of the invention in inhalable form.

Dosages of agents of the invention employed in practicing the present invention will of course vary depending, e.g., on the particular condition to be treated, the effect desired and the mode of administration. In general, suitable daily dosages for oral administration are of the order of 0.01-100 mg/kg.

1. A compound of formula (I) 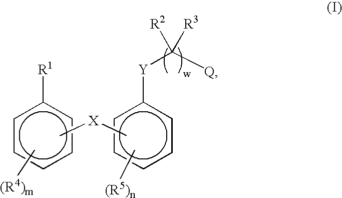
in free or pharmaceutically acceptable salt form, wherein
Q is selected from —C(O)OR⁶, and —C(O)NR⁷R⁸;
R¹ is selected from OH, R^{1a}S—, R^{1a}O— and R^{1a}NR⁹—, wherein R^{1a} is 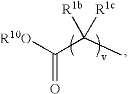
wherein R^{1b} and R^{1c} are, independently, H, C₁-C₈-alkyl, or together with the carbon atom to which they are attached form a divalent C₃-C₈-cycloaliphatic group;
R² and R³ are, independently, H, C₁-C₈-alkyl, or together with the carbon atom to which they are attached form a divalent C₃-C₈-cycloaliphatic group;
R⁴ and R⁵ are, independently, halogen, C₁-C₈-alkyl, C₁-C₈-haloalkyl, a C₃-C₁₅-carbocyclic group, nitro, cyano, C₁-C₈-alkylsulfinyl, C₁-C₈-alkylsulfonyl, C₁-C₈-haloalkylsulfonyl, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, C₁-C₈-alkylamino, di(C₁-C₈-alkyl)amino, SO₂NH₂, (C₁-C₈-alkylamino)sulfonyl, di(C₁-C₈-alkyl)aminosulfonyl, aminocarbonyl, C₁-C₈-alkylaminocarbonyl, di(C₁-C₈-alkyl)aminocarbonyl or a 4- to 10-membered heterocyclic group having one or more heteroatoms selected from the group consisting of oxygen, nitrogen and sulphur;
R⁶ is selected from H, C₁-C₈-alkyl, C₃-C₅-cycloalkyl, C₁-C₈-alkyl(C₆-C₁₅-aromatic carbocyclic group) and C₆-C₁₅-aromatic carbocyclic group;
R⁷ is H or C₁-C₈-alkyl;
R⁸ is C₃-C₁₅-cycloalkyl;
R⁹ and R¹⁰ are, independently selected from H, C₁-C₈-alkyl, C₃-C₁₅-cycloalkyl, C₁-C₈-alkyl(C₆-C₁₅-aromatic carbocyclic group), and C₆-C₁₅-aromatic carbocyclic group;
X is —CH₂—, —CH(C₁-C₈-alkyl)-, —CO—, —CH(OH)—, —CH(OC₁-C₈-alkyl)-, —C(halogen)₂-, —O—, —S—, —SO— or —SO₂—;
Y is —O—, —S—, —CH₂— or —NR¹¹(C₁-C₈alkyl)-;
R¹¹ is selected from H, C₁-C₈-alkyl, C₃-C₁₅-cycloalkyl, C₁-C₈-alkyl(C₆-C₁₅-aromatic carbocyclic group), and a C₆-C₁₅-aromatic carbocyclic group;
m and n are each, independently, an integer selected from 0-3;
v is an integer selected from 1-3; and
w is an integer selected from 0-3, provided that when Q is H, w is an integer selected from 1-3, with the proviso that said compound of formula (I) is not [2-(2-carboxymethoxy-5-chloro-benzyl)-4-chloro-phenoxy]-acetic acid, [2-(2-carboxymethoxy-5-methyl-benzyl)-4-methyl-phenoxy)-acetic acid, 2-{2-[2-(1-carboxy-1-methyl-ethoxy)-5-chloro-benzyl)-4-chloro-phenoxy}-2-methyl-propionic acid, 2-{2-[((1-carboxyethoxy)-5-chloro-3-methyl-benzyl]-4-chloro-6-methyl-phenoxy}-propionic acid, 3′,3′-[methylenebis[(4-methyl-2,1-phenylene)bis-propanoic acid, 2,2′-[methylenebis[4-(1,1-dimethylethyl)-2,1-phenylene]oxy]]bis-acetic acid, diethyl ester, 2,2′-[methylenebis[(3,4,6-trichloro-2,1-phenylene)oxy]]bis-acetic acid, 4-[4-chloro-2-[(5-chloro-2-hydroxyphenyl)methyl]phenoxy]-butanoic acid, monosodium salt, 4-[4-chloro-2-[(4-chloro-2,1-phenylene)oxy]]-butanoic acid, disodium salt, [4-chloro-2-[(4-chloro-2-hydroxyphenyl)methyl]phenoxy]-acetic acid, 2,2′-[methylenebis[(4-chloro-2,1-phenylene)oxy]]bis-acetic acid, [thiobis[(4,6-dichloro-o-phenylene)oxy]di-acetic acid, 3,3′-[methylenebis[(3,4,6-trichloro-o-phenylene)oxy]]di-propionic acid, 2,2′-[methylenebis[(4-methyl-2,1-phenylene)oxy]]bis-acetic acid, or 2,2′-[methylenebis[(4-methyl-2,1-phenylene)oxy]]bis-acetic acid, diethyl ester.

2. A compound of formula (I) according to claim 1, in free or pharmaceutically acceptable salt form,
wherein
Q is selected from —C(O)OR⁶ and —NR⁷C(O)R⁸;
R¹ is selected from OH and 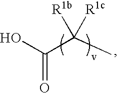
where R^{1b} and R^{1c} are, independently, selected from H and C₁-C₈-alkyl;
R² and R³ are H;
R⁴ and R⁵ are, independently, selected from H, halogen, nitro, and C₁-C₈-alkyl;
X is —CH₂—, —CH(C₁-C₈-alkyl)-, —CO—, —CH(OH)—, —CH(OC₁-C₈-alkyl)-, —C(halogen)₂-, —O—, —S—, —SO— or —SO₂—;
Y is —O—, —S—, —CH₂— or —NR¹¹(C₁-C₁₀-alkyl)-, where R¹¹ is selected from H, C₁-C₈-alkyl, C₃-C₁₅-cycloalkyl, C₁-C₈-alkyl(C₆-C₁₅-aromatic carbocyclic group), and C₆-C₁₅-aromatic carbocyclic group;
m and n are 1;
v is an integer selected from 1-3; and
w is 1.

3. A compound according to claim 1 in free or pharmaceutically acceptable salt form, wherein the compound is of formula (Ia) 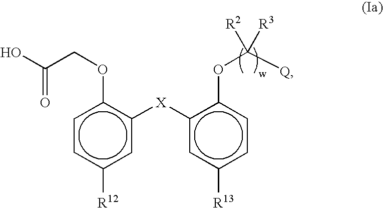
wherein
Q is selected from —C(O)OR⁶, and —C(O)NR⁷R⁸;
R² and R³ are H;
R⁶ is H or C₁-C₈-alkyl;
R⁷ is H;
R³ is C₃-C₁₅ cycloalkyl;
R¹² and R¹³ are, independently, H, halogen, nitro, or C₁-C₃-alkylsulfonyl;
X is —CH₂—, S, —SO— or —SO₂—; and
w is 1.

4. A compound according to claim 1 selected from:
[4-chloro-2-(5-chloro-2-methoxycarbonylmethoxy-benzyl)-phenoxy]-acetic acid;
[4-chloro-2-(5-chloro-2-ethoxycarbonyl methoxy-benzyl)-phenoxy]-acetic acid;
[4-chloro-2-(5-chloro-2-isobutoxycarbonyl methoxy-benzyl)-phenoxy]-acetic acid;
[4-chloro-2-(5-chloro-2-isopropoxycarbonylmethoxy-benzyl)-phenoxy]-acetic acid,
[4-bromo-2-(5-bromo-2-carboxymethoxy-benzyl)-phenoxy]-acetic acid;
[4-chloro-2-(5-chloro-2-cyclopropylcarbamoylmethoxybenzyl)phenoxy]-acetic acid;
[2-(2-carboxymethoxy-5-chloro-benzyl)-phenoxy]-acetic acid;
[4-fluoro-2-(5-fluoro-2-carboxymethoxy-benzyl)-phenoxy]-acetic acid;
[2-(2-carboxymethoxy-5-fluoro-benzyl)-4-fluoro-phenoxy]-acetic acid;
{4-chloro-2-[2-carboxymethoxy-5-chloro-phenylsulfanyl]-phenoxy}-acetic acid;
{4-chloro-2-[benzenesulfonyl-5-chloro-2-carboxymethoxy]-phenoxy}-acetic acid;
4-[2-(2-carboxymethoxy-5-chloro-benzyl)-4-chloro-phenoxy]-butyric acid;
[2-(2-carboxymethoxy-5-chloro-benzyl)-4-nitro-phenoxy]-acetic acid;
2-[2-(2-carboxymethoxy-5-chloro-benzyl)-4-chloro-phenoxy]-2-methylpropionic acid;
3-[2-(2-carboxymethoxy-5-chloro-benzyl)-phenyl]-propionic acid;
[2-(2-carboxymethoxy-5-chloro-benzoyl)-4-chloro-phenoxy]-acetic acid; and
[2-(2-carboxymethoxy-5-methanesulfonyl-benzyl)-4-chloro-phenoxy]-acetic acid.

5. A compound according to claim 1 for use as a pharmaceutical.

6. Pharmaceutical compositions comprising a compound according to claim 1.

7. The use of a compound according to claim 1 in the manufacture of a medicament for treatment of a disease mediated by the CRTh₂ receptor.

8. The use of a compound according to claim 1 in the manufacture of a medicament for treatment of an inflammatory or allergic condition, particularly an inflammatory or obstructive airways disease.

9. A process for the preparation of compounds of formula (I) as defined in claim 1, in free or pharmaceutically acceptable salt form, which comprises the steps of:
(i) (A) for the preparation of compounds of formula (I),
wherein
R¹ is R^{1a}S—, R^{1a}O— or R^{1a}NR⁹, where R^{1a} is 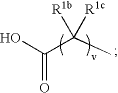
and
all other symbols are as hereinbefore defined,
cleaving an ester group —COOR¹⁰ in a compound of formula (I),
wherein
R¹ is R^{1a}S—, R^{1a}O— or R^{1a}NR⁹,
where
R^{1a} is 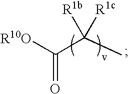
and
R¹⁰ is selected from C₁-C₈ alkyl, C₃-C₁₅-cycloalkyl, C₁-C₈-alkyl(C₆-C₁₅-aromatic carbocyclic group) and C₆-C₁₅-aromatic carboxylic group; and
all other symbols are as hereinbefore defined;
(B) for the preparation of compounds of formula (I),
wherein
R¹ is OH;
Q is —COOH; and
all other symbols are as hereinbefore defined,
appropriately cleaving an ester group in a compound of formula (I),
wherein
Q is —COOR⁶;
R¹ is OH;
R⁶ is C₁-C₈-alkyl, C₃-C₅-cycloalkyl, C₁-C₈-alkyl(C₆-C₁₅-aromatic carbocyclic group), or C₆-C₁₅-aromatic carboxylic group; and
all other symbols are as hereinbefore defined; or
(C) for the preparation of compounds of formula (I),
wherein
Q is COOR⁶ or —C(O)NR⁷R⁸;
R⁶ is C₁-C₈-alkyl, C₃-C₅-cycloalkyl, C₁-C₈-alkyl(C₆-C₁₅-aromatic carbocyclic group), and C₆-C₁₅-aromatic carboxylic group; and
R⁷ and R⁸ are, as hereinbefore defined, appropriately esterifying or amidifying a compound of formula (I), where Q is —COOH; and
(ii) recovering the resultant compound of formula (I), in free or pharmaceutically acceptable salt form.

10. A compound of formula (III) 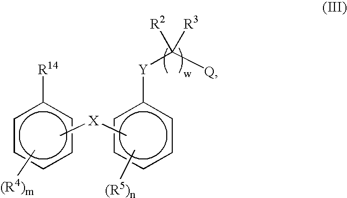
in free or pharmaceutically acceptable salt form, wherein
Q is —C(O)OR⁶,
R² and R³ are, independently, H, C₁-C₈-alkyl or together with the carbon atom to which they are attached form a divalent C₃-C₈-cycloaliphatic group;
R⁴ and R⁵ are, independently, halogen, C₁-C₈-alkyl, C₁-C₈-haloalkyl, a C₃-C₁₅-carbocyclic group, nitro, cyano, C₁-C₈-alkylsulfonyl, C₁-C₈-alkylcarbonyl, C₁-C₈-alkoxycarbonyl, C₁-C₈-alkoxy, C₁-C₈-haloalkoxy, carboxy, carboxy-C₁-C₈-alkyl, amino, C₁-C₈-alkylamino, di(C₁-C₈-alkyl)amino, SO₂NH₂, (C₁-C₈-alkylamino)sulfonyl, di(C₁-C₈-alkyl)aminosulfonyl, aminocarbonyl, C₁-C₈-alkylaminocarbonyl, di(C₁-C₈-alkyl)aminocarbonyl or a 4- to 10-membered heterocyclic group having one or more heteroatoms selected from the group consisting of oxygen, nitrogen and sulphur;
R⁶ is selected from H, C₁-C₈-alkyl, C₃-C₅-cycloalkyl, C₁-C₈-alkyl(C₆-C₁₅-aromatic carbocyclic group) and C₆-C₁₅-aromatic carbocyclic group; X is —CH₂—, —CO—, —CH(OH)—, —CH(OC₁-C₈-alkyl)-, —C(halogen)₂-, —O—, —S—, —SO— or —SO₂—;
Y is —O—, —S—, —CH₂— or —NR¹¹(C₁-C₈-alkyl);
R¹¹ is selected from H, C₁-C₈-alkyl, C₃-C₅-cycloalkyl, C₁-C₈-alkyl(C₆-C₁₅-aromatic carbocyclic group) and C₆-C₁₅-aromatic carbocyclic group;
R¹⁴ is —(CR^{1b}R^{1c})ₚCN, wherein R^{1b} and R^{1c} are, independently, H or C₁-C₈-alkyl;
m and n are each, independently, an integer selected from 0-3;
p is an integer selected from 0-2; and
w is an integer selected from 0-3.
